(19) United States
(12) Patent Application Publication
(10) Pub. No.: US 2019/0358327 A1
(43) Pub. Date: Nov. 28, 2019

(54) BIOPHOTONIC COMPOSITIONS COMPRISING LICHEN EXTRACT AND THEIR USE TO TREAT SKIN DISORDERS

(71) Applicant: KLOX TECHNOLOGIES INC., Laval (CA)

(72) Inventors: David OHAYON, Dollard-des-Ormeaux (CA); Paschalis PASCHALAKIS, Dublin (IE); Remigio PIERGALLINI, San Benedetto del Tronto (IT); Nikolaos LOUPIS, Athens (GR)

(21) Appl. No.: 16/472,643

(22) PCT Filed: Dec. 22, 2017
(86) PCT No.: PCT/CA2017/051597

§ 371 Date: Jun. 21, 2019

Related U.S. Application Data
(60) Provisional application No. 62/438,895, filed on Dec. 23, 2016.

(51) Int. Cl. A61K 41/00; A61K 36/09; A61P 17/10

(52) U.S. Cl. CPC A61K 41/00; A61P 17/10; A61K 36/09

(57) ABSTRACT

The present disclosure provides biophotonic compositions comprising a lichen extract and a carrier medium and methods and uses thereof.

### CROSS-REFERENCE TO RELATED APPLICATION

This application claims the benefit of and priority to U.S. provisional patent application No. 62/438,895, filed Dec. 23, 2016, the content of which is herein incorporated in its entirety by reference.

### FIELD OF TECHNOLOGY

The present technology relates to biophotonic compositions comprising a lichen extract and a carrier medium and methods and uses thereof.

### BACKGROUND

Acne is the eighth most prevalent disease worldwide where it is estimated to affect 9.4% of the global population. Acne vulgaris, or acne, is a common condition with a wide range of potential harms and associated costs. The condition includes symptomatic discomfort, scarring, emotional and psychosocial distress, occupational consequences and potential psychiatric disturbances including depression and suicide (Tan, J. K. L, and Bhate, K., British Journal of Dermatology 2015, 172 (Suppl. 1): 3-12). It was estimated that over $1 billion is spent each year in the US on acne related health care visits and acne therapies (Nelson et al., Frontiers in Pharmacology, November 2016, Vol. 7, Article 425: 1-425).

Propionibacterium acnes has been implicated in the pathogenesis of acne, where it is a condition that occurs when the hair follicles become plugged with oil and dead skin, and usually appears on the face, neck, chest back and shoulders. Current treatments fall into either two categories: topical or oral treatment. The common topical treatments include benzoyl peroxide, retinoids, and antibiotics (i.e., erythromycin or clindamycin) and common oral treatments include retinoids and antibiotics (i.e., tetracycline and macrolides).

Phototherapy is recognized as having a wide range of applications in both the medical and cosmetic fields. For example, phototherapy has been used to disinfect target sites as an antimicrobial treatment, to promote wound healing, and for skin rejuvenation. Phototherapy has also been used to control or treat skin conditions such as acne, psoriasis, rosacea and scarring. There are devices, such as a home facial mask, that may be used in the treatment of acne. In low level light therapy (phototherapy), the skin is exposed to low intensity LED or laser light.

There is thus a need in the field to provide improved compositions and methods useful in phototherapy. There is also a need in the field for the use of phototherapy on a daily basis at home without the need of a physician or nurse.

### SUMMARY OF TECHNOLOGY

According to one embodiment, the technology of the present disclosure provides biophotonic compositions for use in biophotonic therapy. The biophotonic compositions of the present disclosure can be used in combination with a low intensity light therapy device for the treatment of a skin condition, such as acne or rosacea, or for preventing or treating scarring. In some embodiments, a biophotonic composition of the present disclosure comprises at least one lichen extract and a carrier medium. In some embodiments, the biophotonic composition comprises a lichen extract comprising at least one chromophore that can be excited by low intensity light. In some implementations of this embodiment, the biophotonic compositions of the present disclosure have both antibacterial and anti-inflammatory properties. In some other implementations, the biophotonic composition is a cosmetic composition.

The present disclosure provides improved biophotonic compositions and their methods and uses in biophotonic therapy or phototherapy. In some embodiments, the biophotonic composition of the present disclosure comprises a lichen extract and a carrier medium. In certain such embodiments, the lichen extract comprises at least one chromophore (i.e., at least one lichen-derived chromophore).

In some embodiments, the lichen extract is derived from lichen selected from the group consisting of Rhizocarpon geographicum, Xanthoparmelia scabrosa, Lecidella elaeochroma, Cetraria islandica, and Xanthoria parietina. In some embodiments, the lichen extract is derived from Rhizocarpon geographicum. In some embodiments, the lichen extract is derived from Xanthoparmelia scabrosa. In some embodiments, the lichen extract is derived from Lecidella elaeochroma. In some embodiments, the lichen extract is Cetraria islandica. In some embodiments, the lichen extract is derived from Xanthoria parietina.

In some embodiments, the carrier medium comprises one or more of a hydrophilic polymer, a hygroscopic polymer, or a hydrated polymer, or combinations thereof. In some embodiments, the carrier medium is polyanionic in charge character. In some embodiments, the carrier medium comprises carboxylic functional groups. In some embodiments, the carrier medium comprises a polymer having from 2 to 7 carbon atoms per functional group.

In some embodiments, the carrier medium comprises one or more of a synthetic polymer selected from a vinyl polymer, a polyoxyethylene-polyoxypropylene copolymer, poly(ethylene oxide), an acrylamide polymer, and derivatives or salts of any of the foregoing, and combinations thereof. In some embodiments, the carrier medium comprises one or more of a vinyl polymer selected from polyacrylic acid, polymethacrylic acid, polyhydroxyethyl methacrylate, polyvinyl pyrrolidone, and polyvinyl alcohol. The carrier medium may comprise a carboxy vinyl polymer or a carbomer obtained by polymerization of acrylic acid. In certain such embodiments, the carboxy vinyl polymer or carbomer may be crosslinked.

In some embodiments, the carrier medium comprises a vinyl polymer selected from Carbopol® 940, Carbopol® 980, ETD 2020 NF, Carbopol® 1382 Polymer, 71G NF, 971P NF, 974P NF, 980 NF, 981 NF, 5984 EP, ETF 2020 NF, ultrez 10 NF, ultrez 20, ultrez 21, 1342 NF, 934 NF, 934P NF, 940 NF, and 941 NF, and combinations thereof.

In some embodiments, the carrier medium comprises 2-hydroxyethyl methacrylate (HEMA) either alone or in addition to another carrier. In some embodiments, the 2-Hydroxyethyl methacrylate (HEMA) is added to the carrier medium in the form of microspheres or in a further physically reduced form such as in a finely ground particulate form or in a pulverized, powder form. In some embodiments, the carrier medium comprises a polyacrylic acid polymer cross-linked with alkyl acrylate or allyl pentaerythritol.

In some embodiments, the carrier medium comprises one or more polymer. In some embodiments, the polymer is present in an amount of between about 0.05% and about 5% by weight of the composition, or between about 0.1% and about 2.5%, or between about 0.1% and about 2%, or between about 0.5% and about 2.5%, or between about 0.5% and about 2% by weight of the total composition. In some embodiments, the polymer is present in an amount of between about 0.05% and about 5% by weight of the total composition, or between about 0.1% and about 2.5%, or between about 0.1% and about 2%, or between about 0.5% and about 2.5%, or between about 0.5% and about 2% by weight of the total composition.

In some embodiments, the carrier medium comprises one or more protein-based polymer. In some embodiments, the protein-based polymer is one or more of elastin, gelatin, and collagen. In some embodiments, the carrier medium comprises gelatin. In certain such embodiments, the gelatin is present in an amount of equal to or more than about 4% by weight of the total composition, such as 4% by weight of the total composition. In other embodiments, the carrier medium comprises collagen. In certain such embodiments, the collagen is present in an amount equal to or more than about 5% by weight of the total composition, such as 5% by weight of the total composition.

In certain embodiments, the carrier medium comprises one or more natural or synthetic polysaccharide. In some embodiments, the polysaccharide is selected from sodium hyaluronate, starch, chitosan, chitin, agar, alginates, xanthan, carrageenan, guar gum, gellan gum, hydroxypropyl cellulose, carboxymethyl cellulose, pectin, locust bean gum, and combinations thereof.

In some embodiments, the carrier medium comprises at least one glycol. In some embodiments, the glycol is selected from ethylene glycol, propylene glycol, and combinations thereof.

In some embodiments, the biophotonic composition is in the form of an emulsion, a cream, a serum, a lotion, a solution, or a gel. In some embodiments, the biophotonic composition may be absorbed into skin.

In some embodiments, the carrier medium comprises a pharmaceutically acceptable, dermatologically acceptable, or cosmetically acceptable medium.

In some aspects, the biophotonic compositions of the present disclosure comprise a lichen extract, wherein the lichen extract comprises at least one chromophore (i.e., a lichen-derived chromophore). In some embodiments, the chromophore is a fluorescent chromophore. In certain embodiments, the at least one chromophore may be in the form of a molecular complex that conserves the photochemical properties of the at least one chromophore. In certain embodiments, the chromophore or chromophores that are derived from at least one lichen source conserve their photochemical properties. In some embodiments, the at least one chromophore or molecular complex is extracted and/or isolated and/or purified from the at least one lichen source through methods and techniques known in the art. In some implementations, the at least one chromophore or molecular complex is in a form that is “purified”, “isolated” or “substantially pure”. The chromophore(s) or molecular complex(es) is said to be “purified”, “isolated” or “substantially pure” when it or they are separated from the components that naturally accompany them. Typically, a compound is considered “purified”, “isolated”, or “substantially pure” when it comprises at least 25%, at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, by weight, of the total composition in a sample.

In some embodiments, the lichen extract is selected such that its photoexcitation by light, such as a low intensity light, can result in the subsequent emission of light (e.g., fluorescence). In some embodiments, the lichen-derived chromophore is selected such that its photoexcitation by light, such as a low intensity light, can result in the subsequent emission of light (e.g., fluorescence). It is believed that low light intensities have a rejuvenating and therapeutic effect on tissues such as skin. This may increase collagen synthesis in the tissue and/or have an antimicrobial effect. Photoexcitation is preferably not accompanied by concomitant generation of heat. In certain embodiments, the photoexcitation of the lichen extract does not result in tissue damage. In certain embodiments, the photoexcitation of the lichen-derived chromophore does not result in tissue damage.

In some embodiments, the chromophore can be photoactivatable by low intensity light. In some embodiments, the chromophore or molecular complex absorbs and/or emits light within the visible range. In some embodiments, the chromophore or molecular complex absorbs and/or emits light within the green, orange and yellow portions of the electromagnetic spectrum. In some embodiments, the chromophore or molecular complex absorbs and/or emits light within the range from about 400 to about 700 nm. In some embodiments, the chromophore or any of the multiplicity of chromophores is derived from a lichen extract.

In some embodiments, the lichen extract is present in an amount of from about 0.1% to about 10% by weight of the total composition, or from about 0.1% to about 5% by weight of the total composition, or about 5% by weight of the total composition, or about 4% by weight of the total composition, or about 3% by weight of the total composition, or about 2% by weight of the total composition, or about 1% by weight of the total composition. In some embodiments, the at least one chromophore is present in an amount of from about 0.0001% to about 10% by weight of the total composition, or from about 0.0001% to about 2% by weight of the total composition.

In some embodiments, upon exposure to light, the composition emits at least 1.25×, 1.5×, 1.75× or 2× more red, yellow and/or orange light than a composition lacking the lichen extract. In other embodiments, upon exposure to light, the composition emits at least 5×, 10× or 20× more red, yellow and/or orange light than a composition lacking the lichen extract.

In some aspects of the disclosure, there is provided a kit comprising the biophotonic compositions of this disclosure.

In some aspects of the disclosure, the biophotonic compositions of this disclosure are used for treatment of a skin condition. In some embodiments, the skin condition is selected from acne, eczema, psoriasis, and dermatitis. In some embodiments, the biophotonic compositions of this disclosure are used for skin rejuvenation and conditioning. In some embodiments, the biophotonic compositions of this disclosure are used for preventing and treating scarring.

In some aspects of the disclosure, the biophotonic compositions of this disclosure are used for reducing inflammation. In certain embodiments, the biophotonic compositions of this disclosure are used for an antibacterial and/or bacteriostatic effect. In certain embodiments, the biophotonic compositions of this disclosure are used for reducing bacterial infection. In certain such embodiments, the inflammation is associated with P. acnes infection.

In some aspects of the disclosure, this disclosure provides a method for biophotonic treatment of a skin disorder comprising:
applying a biophotonic composition to a target skin tissue, wherein the biophotonic composition comprises at least one lichen extract and a carrier medium; and
illuminating said biophotonic composition with low intensity light.

In some embodiments, the skin disorder is selected from acne, eczema, psoriasis, and dermatitis.

In some aspects of the disclosure, this disclosure provides a method for biophotonic treatment of acne comprising:
applying a biophotonic composition to a target skin tissue, wherein the biophotonic composition comprises at least one lichen extract and a carrier medium; and
illuminating said biophotonic composition with low intensity light.

In some aspects of the disclosure, this disclosure provides a method for promoting skin rejuvenation comprising:
applying a biophotonic composition to a target skin tissue, wherein the biophotonic composition comprises at least one lichen extract and a carrier medium; and
illuminating said biophotonic composition with low intensity light.

In some aspects of the disclosure, there is provided a biophotonic composition prepared by the following steps:
(a) Pulverizing at least one lichen source to provide a semi-fine, homogenous powder;
(b) Adding at least an equivalent amount (such as the same amount, or one-fold excess, two-fold excess, five-fold excess, ten-fold excess, or twenty-fold excess) of propylene glycol by weight to said homogenous powder;
(c) Stirring the resulting solution from step (b) at low speed for at least five days (such as ten days, fifteen days, or twenty days);
(d) Filtering the solution of step (c) to obtain a lichen extract comprising at least one chromophore; and
(e) Combining the lichen extract with a carrier medium.

In some embodiments, the biophotonic composition is prepared in dark.

In some embodiments, the biophotonic compositions as defined herein further comprise a chromophore-protecting agent such as, but not limited to, a buffer, a salt, and a solvent that preserves the photochemical activity or property of the chromophore(s).

In some embodiments, the lichen extract is derived from lichen selected from Rhizocarpon geographicum, Xanthoparmelia scabrosa, Lecidella elaeochroma, Cetraria islandica, and Xanthoria parietina. In some embodiments, the lichen extract is derived from Rhizocarpon geographicum. In some embodiments, the lichen extract is derived from Xanthoparmelia scabrosa. In some embodiments, the lichen extract is derived from Lecidella elaeochroma. In some embodiments, the lichen extract is derived from Cetraria islandica. In some embodiments, the lichen extract is derived from Xanthoria parietina.

In certain embodiments, the light that may be useful for illumination of the biophotonic composition as defined herein is a continuous light. In other embodiments, the light that may be useful for illumination of the biophotonic composition as defined herein is a modulated light such as a pulsed light. In certain such embodiments, the light source that may be useful for illumination of the biophotonic composition as defined herein is a light-emitting diode (LED) or an array of LEDs. In some implementations, the light that may be useful for illumination of the biophotonic composition is a low intensity light.

In some aspects, the present disclosure generally relates to biophotonic compositions for treating a skin condition, such as acne, rosacea, and scarring, and/or for maintaining and/or improving a skin condition, wherein said compositions comprise a lichen extract, such as lichen extract of Rhizocarpon geographicum, Xanthoparmelia scabrosa, Lecidella elaechroma, Cetraria islandica, or Xanthoria parietina. In certain such aspects, the lichen extract comprises, e.g., 4% w/w of the total composition. In other aspects, the present disclosure generally relates to cosmetic use and/or methods for treating a skin condition, such as acne, rosacea, and scarring, and/or for maintaining and/or improving a skin condition, comprising administering a composition of the disclosure, e.g., a composition comprising a lichen extract, such as lichen extract of Rhizocarpon geographicum, Xanthoparmelia scabrosa, Lecidella elaechroma, Cetraria islandica, or Xanthoria parietina.

In some aspects, there is a provided a composition for cosmetic use comprising a lichen extract and a carrier medium in combination with a low intensity light therapy device for the treatment of a skin condition such as acne, rosacea, or for preventing or treating scarring. The disclosure contemplates that any of the embodiments set forth below can be combined with each other or with any of the aspects or embodiments set forth above, or otherwise set forth herein.

### BRIEF DESCRIPTION OF THE DRAWINGS

FIG. 1 illustrates a graph showing the absorbance spectra of lichen extracts in a propylene glycol solution or in Composition A (Example 3; Composition A is specified in Example 2);

FIG. 2 illustrates a graph showing the emission spectra of lichen extracts in a propylene glycol solution or in Composition A (Example 3);

FIG. 3 illustrates a graph showing the antibacterial activities of the 4% AXSE¹ lichen (lichen derived from Xanthoparmelia scabrosa) in Composition A+/−light (illumination with a low intensity light source A or a low intensity light source B for 10 minutes) on P. acnes using a dilution neutralization method. Lichen extract (Xanthoparmelia scabrosa) was prepared with 1 gram of lichen powder in 10 grams of propylene glycol (Example 4);

FIG. 4 illustrates a graph showing the anti-bacterial activities of a BioPhotonic (BP) solution according to an embodiment of the present technology on P. acnes+/−light (illumination with a low intensity light source A) using a dilution neutralization method (Example 5);

FIGS. 5 and 6 show pictures of clinical assessment results of using a BioPhotonic (BP) lotion of the present disclosure with illumination by a low intensity light source in to ameliorate an acne condition in a patient versus use of illumination with the low intensity light source alone. Patients were treated once per day following a split face protocol in which the BioPhotonic lotion was applied to only one side of the patient's face. Both sides of the face were then concomitantly illuminated with a low intensity light source A. FIG. 5 shows the treatment results on a male patient and FIG. 6 shows the treatment results on a female patient (Example 6); and

FIG. 7 illustrates graphs showing the anti-bacterial activities of a BioPhotonic (BP) solution according to another embodiment of the present technology on P. acnes+/−light (illumination with a low intensity light source A) using a dilution neutralization method (Example 7).

### DETAILED DESCRIPTION

### (1) Overview

Skin is the largest organ of the body, accounting for 12% to 16% of body weight. Skin is made up of two main layers that cover a third fatty layer. The outer layer is the epidermis, and second layer beneath epidermis is the dermis. Under these two skin layers is a fatty layer of subcutaneous tissue. With age the amount of subcutaneous (under-the-skin) fat is reduced resulting in a looser look to the skin. Skin changes, such as wrinkles and sagging skin, are among the most visible signs of aging.

In some embodiments, the present disclosure provides compositions and methods which may be useful for cosmetic use, the treatment/maintenance of a skin condition, and/or for skin rejuvenation. In particular, the compositions of the present disclosure comprise a lichen extract comprising at least one chromophore and a suitable carrier medium, e.g., a dermatologically acceptable carrier, wherein the chromophore is photoexcitable or photoactivatable. The compositions of the present disclosure may be considered to be cosmetic biophotonic compositions.

### (2) Definitions

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

It is convenient to point out here that “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

“Direct light” means light irradiation directly from a light source, such as from a lamp, mobile device, screen, sun etc.

“Biophotonic” means the generation, manipulation, detection and application of photons in a biologically relevant context. In other words, biophotonic compositions exert their physiological effects primarily due to the generation and manipulation of photons, for example, by absorbing photon to emit photons or to transfer energy, for example, by absorbing photons to emit photons or to transfer energy. “Biophotonic composition” is a composition as described herein that may be activated or excited by radiant energy to generate photons for therapeutic effect.

“Photoexcitable” or “photoactive” in relation to a chromophore is meant that the molecules of the chromophore are able to absorb radiant energy within the medium of the dermatologically acceptable carrier. The excited state is referred to herein, interchangeably, as ‘photoexcited’ or ‘photoactivated’. A photoactivated chromophore may transition to an excited state leading to the emission of the absorbed energy as light, e.g., fluorescence, or to transfer of the absorbed energy to other molecules.

“Topical composition” means a composition to be applied to body surfaces, such as the skin, mucous membranes, vagina, oral cavity, lesions, scars, surgical wound sites, and the like. A topical composition may be in the form of, including, but not limited to, a cream, emulsion, gel, ointment, lotion, levigate, solution, paste, bioadhesive, salve, milk, impregnated pad, spray, suspension, foam, or the like.

Terms “chromophore”, “photoactivating agent”, “photoactivator” and “light-absorbing molecule” are used herein interchangeably. A chromophore means a chemical compound, moiety, or complex, when contacted by light irradiation, is capable of absorbing the light. The chromophore can undergo photoexcitation and can then emit its energy as light (e.g., fluorescence).

The term “actinic light” is intended to mean light energy emitted from a specific light source (e.g., lamp, LED, laser or sunlight) and capable of being absorbed by matter (e.g., the chromophore(s) or photoactivator(s)). The expression “actinic light” and the term “light” are used herein interchangeably. In some embodiments, the actinic light is visible light.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

### (3) Biophotonic Topical Compositions Comprising Lichen Extract

In some embodiments, the present disclosure provides biophotonic compositions such as cosmetic biophotonic compositions. Biophotonic compositions are, in a broad sense, compositions that can be activated by light of specific wavelengths. A biophotonic composition according to various embodiments of the present disclosure comprises a lichen extract and a carrier medium, wherein the lichen extract comprises at least one chromophore. These compositions of this disclosure comprise a lichen extract which can be excited by light to emit light of a different wavelength than the absorbed light, and/or to cause photochemical activation of other agents contained in the composition or in the tissue.

When a chromophore absorbs a photon of a certain wavelength, it becomes excited. This is an unstable condition and the molecule tries to return to the ground state, giving away the excess energy. For some chromophores, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. This is called the Stokes' shift. In a biophotonic composition, much of this energy may be transferred to the other components of the biophotonic composition or to the treatment site directly.

Without being bound to theory, it is thought that fluorescent light emitted by photoactivated chromophores may have therapeutic properties due to its femto-, pico-, or nano-second emission properties which may be recognized by biological cells and tissues, leading to biomodulation. Furthermore, the emitted fluorescent light has a longer wavelength and hence a deeper penetration into the tissue than the activating light. Irradiating tissue with such a broad range of wavelengths, including in some embodiments, the activating light which passes through the composition, may have different and complementary effects on the cells and tissues. In other words, lichen extracts which contain at least one chromophore are used in the biophotonic compositions of the present disclosure for cosmetic and/or therapeutic effect on tissues.

The biophotonic compositions of the present disclosure are for topical uses. The biophotonic compositions of the present disclosure may be described based on the components making up the composition. Additionally, or alternatively, the compositions of the present disclosure have functional and structural properties and these properties may also be used to define and describe the compositions.

The compositions according to the present disclosure are provided in a carrier medium, such as a dermatologically acceptable carrier. In some embodiments, the carrier is more or less fluid and may have the appearance of a clear, white, or colored cream, an ointment, a milk-like liquid, a lotion, a serum, a paste, a solution, or foam. The composition can optionally be sprayed on the skin, and in this case be in the form of an aerosol or a liquid. Alternatively, the composition is spreadable on the skin, and in this case can be in the form of a lotion or a gel.

The composition according to the present disclosure can in particular be provided in the form of a product for caring for the skin of the face or body, the lips, the eyelashes, the eyebrows or the scalp; or of an aftershave gel or lotion; or of a depilatory cream; or of a moisturizer or a barrier cream.

Individual components of the biophotonic compositions according to various embodiments of the present disclosure, including lichen extracts, carrier mediums, and other optional ingredients, are detailed below.

(f) Lichens Extracts

The compositions of the present disclosure comprise one or more lichen extracts. Lichen are symbiotic associations between fungi (mycobionts), unicellular green algae, bacteria and occasionally cyanobacteria (photobionts). Since fungi are incapable of photosynthesis due to a lack of chlorophyll, they rely on algae, bacteria or cyanobacteria which can undergo photosynthesis to produce food. Lichens grow on a wide range of substrates including the bark of trees, soil, rocks, and deadwood.

Lichen is a composite organism wherein the photobionts live symbiotically with the mycobiants among the hyphae of the mycobiant. The body of lichens (thallus) is structurally and functionally different than either the mycobiont or photobiont living separately. The growth forms of lichen are dependent on both the mycobiont and the photobiont. Lichen are used as a source of food, dyes, and traditional medicine.

For the preparation of the biophotonic compositions of the disclosure, the following methodology was utilized. The lichen extract, e.g., Rhizocarpon geographicum lichen extract, was prepared by crushing the lichen into a semi-fine, homogeneous powder using a kitchen blender device. Thereafter, a suitable amount, e.g., 1 gram, 2 grams, 3 grams or more, of the particular lichen were then added to propylene glycol, e.g., 10 mL, 20 mL, 30 mL or more, and the resulting solution was continuously stirred at low speed over the course of several days, e.g., five (5) days, six (6) days, seven (7) days or more, of continuous mixing. At the completion of the stirring period, the resulting product is left unfiltered with the particulate (solid) fraction remaining on the bottom of the mixing container, while samples are withdrawn from the overlying liquid phase for analysis and inclusion into the biophotonic composition. Depending on the species of the lichen, extraction of the lichen may be carried out using different lichen powder to propylene glycol ratio, such as a ratio of 1:10 (wt:wt), 1:5 (wt:wt), 1:4 (wt:wt), 1:3 (wt:wt), 1:2 (wt:wt), or 1:1 (wt:wt).

In some embodiments, the at least one chromophore (i.e., lichen-derived chromophore) is extracted from at least one lichen source. For example, the at least one chromophore (i.e., lichen-derived chromophore) can be extracted from pulverized lichen using an organic solvent such as acetone, benzene, chloroform, ethyl acetate, ethanol, methanol, petroleum ether, propylene glycol, hexane and DMSO. The at least one chromophore (i.e., lichen-derived chromophore) can then be purified by techniques such as column chromatography (reverse phase or silica gel), liquid chromatography, HPLC, thin layer chromatography (TLC), and gel permeation chromatography. The chromophore resulting from the extraction or from the purification can be characterized using techniques such as UV-vis, FTIR, ESI-MS, and HPLC-MS.

The biophotonic compositions of the present disclosure comprise a lichen extract, wherein the lichen extract comprises at least one chromophore, and wherein the chromophore is a lichen-derived chromophore. In some embodiments, the at least one chromophore (i.e., lichen-derived chromophore) absorbs at a wavelength in the range of the visible spectrum, such as at a wavelength of about 380 nm-800 nm, about 380 nm-700 nm, about 400 nm-800 nm, or about 380 nm-600 nm. In some embodiments, the at least one chromophore (i.e., lichen-derived chromophore) absorbs at a wavelength of about 380 nm-600 nm. In some embodiments, the at least one chromophore (i.e., lichen-derived chromophore) absorbs light at a wavelength of about 380 nm-480 nm, about 480 nm-580 nm, about 480 nm-650 nm, about 600 nm-700 nm, about 650 nm-750 nm or about 700 nm-800 nm.

It will be appreciated to those skilled in the art that optical properties of a particular chromophore may vary depending on the chromophore's surrounding medium. Therefore, as used herein, a particular chromophore's absorption and/or emission wavelength (or spectrum) corresponds to the wavelengths (or spectrum) measured in a biophotonic composition of the present disclosure.

In some embodiments, the lichen extracts are selected such that their emitted fluorescent light, on photoactivation, is within one or more of the green, yellow, orange, red, and infrared portions of the electromagnetic spectrum, for example having a peak emission wavelength within the range of about 400 nm to about 800 nm. In some embodiments, the lichen extracts have a peak emission wavelength within the range of about 500 nm to about 700 nm. In some embodiments, the lichen extracts have a peak emission wavelength within the range of about 400 nm to about 600 nm. In some embodiments, the lichen extracts have a peak emission wavelength within the range of about 650 nm to about 750 nm. In certain such embodiments, the emitted fluorescent light has a power density of between about 0.005 mW/cm² and about 10 mW/cm², such as between about 0.5 mW/cm² and about 5 mW/cm². In other embodiments, the emitted fluorescent light has a power density in the micorwatts range, such as a power density of between about 10 μW/cm² and about 50 μW/cm², between about 10 μW/cm² and about 100 μW/cm², between about 10 μW/cm² and about 200 μW/cm², between about 10 μW/cm² and about 300 μW/cm², between about 10 μW/cm² and about 400 μW/cm², between about 10 μW/cm² and about 500 μW/cm², or between about 10 μW/cm² and about 1000 μW/cm².

In some embodiments, the lichen-derived chromophores are selected such that their emitted fluorescent light, on photoactivation, is within one or more of the green, yellow, orange, red, and infrared portions of the electromagnetic spectrum, for example having a peak emission wavelength within the range of about 400 nm to about 800 nm. In some embodiments, the lichen-derived chromophores have a peak emission wavelength within the range of about 500 nm to about 700 nm. In some embodiments, the lichen-derived chromophores have a peak emission wavelength within the range of about 400 nm to about 600 nm. In some embodiments, the lichen-derived chromophores have a peak emission wavelength within the range of about 650 nm to about 750 nm. In certain such embodiments, the emitted fluorescent light has a power density of between about 0.005 mW/cm² to about 10 mW/cm², such as about 0.5 mW/cm² to about 5 mW/cm². In other embodiments, the emitted fluorescent light has a power density in the micorwatts range, such as a power density of between about 10 μW/cm² and about 50 μW/cm², between about 10 μW/cm² and about 100 μW/cm², between about 10 μW/cm² and about 200 μW/cm², between about 10 μW/cm² and about 300 μW/cm², between about 10 μW/cm² and about 400 μW/cm², between about 10 μW/cm² and about 500 μW/cm², or between about 10 μW/cm² and about 1000 μW/cm².

In some embodiments, the lichen extract is obtained from, for example, but not limited to, extracts of Arthoniomycetes, Dothideomycetes, Eurotiomycetes, Lecanoromycetes, Leotiomycetes, Lichinomycetes, Sordariomycetes, Incertae sedis, Basidiomycetes, Urediniomycetes, Coelomycetes, lichens. In some embodiments, the lichen extract is derived from sources including, but not limited to, a species within one of the following genera: Allarthonia, Arthonia, Arthothelium, Coniarthonia, Cryptothecia, Sporostigma, Stirtonia, Chrysothrix, Melaspilea, Bactrospora, Chiodecton, Combea, Cresponea, Dendrographa, Dirina, Enterographa, Hubbsia, Lecanactis, Lecanographa, Mazosia, Opegrapha, Phoebus, Plectocarpon, Reinkella, Roccella, Roccellina, Schismatomma, Schizopelte, Sclerophyton, Sigridea, Syncesia, Arthophacopsis, Llimonaea, Perigrapha, Echinothecium, Cystocoleus, Clypeococcum, Dacampia, Eopyrenula, Polycoccum, Pyrenidium, Collemopsidium, Pyrenocollema, Zwackhiomyces, Melanomma, Taeniolella, Peridiothelia, Epicoccum, Leptosphaerulina, Monoblastiopsis, Phoma, Epigloea, Arthopyrenia, Mycomicrotheli, Didymosphaeri, Lichenostigma, Lichenotheli, Lichenopeltell, Sphaerellothecium, Sphaerulina, Stigmidium, Jarxia, Leptorhaphis, Tomasellia, Parmularia, Myxophora, Raciborskiomyces, Wentiomyces, Pyrenothrix, Protothelenella, Thrombium, Buelliella, Cercidospora, Hassea, Homostegia, Karschia, Monodictys, Mycoglaena, Mycoporellum, Rosellinula, Trematosphaeriopsis, Capronia, Racodium, Acrocordia, Anisomeridium, Monoblastia, Anthracothecium, Distopyrenis, Granulopyrenis, Lithothelium, Polypyrenula, Pyrenula, Pyrgillus, Sulcopyrenula, Requienella, Astrothelium, Bathelium, Campylothelium, Laurera, Polymeridium, Pseudopyrenula, Trypethelium, Celothelium, Mycoporum, Adelococcus, Sagediopsis, Agonimia, Bagliettoa, Bellemerella, Catapyrenium, Clavascidium, Dermatocarpon, Endocarpon, Endococcus, Henrica, Heterocarpon, Heteroplacidium, Involucropyrenium, Lauderlindsaya, Leucocarpia, Merismatium, Muellerella, Neocatapyrenium, Phaeospora, Placidiopsis, Placidium, Placopyrenium, Polyblastia, Psoroglaena, Staurothele, Thelidium, Trimmatothele, Verrucaria, Geisleria, Strigula, Chaenothecopsis, Mycocalicium, Phaeocalicium, Stenocybe, Sphinctrina, Acarospora, Glypholecia, Myriospora, Pleopsidium, Polysporina, Sarcogyne, Anzia, Arthrorhaphis, Gongylia, Biatorella, Acroscyphus, Baculifera, Calicium, Chrismofulvea, Ciposia, Cyphelium, Endohyalina, Texosporium, Thelomma, Tholurna, Candelaria, Candelariella, Candelina, Placomaronea, Cetradonia, Cladonia, Pilophorus, Pycnotheli, Crocynia, Dactylospora, Gypsoplaca, Haematomma, Arctopeltis, Bryonora, Calvitimela, Carbonea, Cladidium, Clauzadeana, Edrudia, Frutidella, Glaucomaria, Lecanora, Lecidella, Miriquidica, Myrionora, Protoparmelia, Psorinia, Pycnora, Pyrrhospora, Ramboldia, Rhizoplaca, Scoliciosporum, Tylothallia, Vainionora, Cecidonia, Hypocenomyce, Lecidea, Myochroidea, Steinia, Loxospora, Catillochroma, Megalaria, Lopezaria, Megalospora, Mycoblastus, Ophioparma, Ahtiana, Alectoria, Allantoparmelia, Allocetraria, Arctocetraria, Arctoparmelia, Asahinea, Brodoa, Bryocaulon, Bryopogon, Bryoria, Bulbothrix, Canoparmelia, Cavernularia, Cetraria, Cetrariastrum, Cetrariella, Cetrelia, Coelocaulon, Cornicularia, Dactylina, Esslingeriana, Evernia, Everniastrum, Flavocetraria, Flavoparmelia, Flavopunctelia, Gowardia, Hypogymnia, Hypotrachyna, Imshaugia, Kaernefeltia, Letharia, Masonhalea, Melanelia, Melanelixia, Melanohalea, Menegazzia, Myelochroa, Neofuscelia, Nesolechia, Nodobryoria, Omphalodium, Omphalora, Paraparmelia, Parmelia, Parmelina, Parmelinopsis, Parmeliopsis, Parmotrema, Parmotremopsis, Phacopsis, Platismatia, Pseudephebe, Pseudevernia, Pseudoparmelia, Punctelia, Relicina, Sulcaria, Tuckermanella, Tuckermannopsis, Usnea, Vulpicida, Xanthoparmelia, Amandinea, Anaptychia, Buellia, Cratiria, Culbersonia, Dermatiscum, Dermiscellum, Dimelaena, Diploicia, Diplotomma, Dirinaria, Gassicurtia, Hafellia, Heterodermia, Hyperphyscia, Mobergia, Phaeophyscia, Phaeorrhiza, Physcia, Physciella, Physconia, Pyxine, Rinodina, Tetramelas, Tornabea, Byssoloma, Calopadia, Fellhanera, Fellhaneropsis, Lopadium, Micarea, Psilolechia, Sporopodium, Szczawinskia, Tapellaria, Amygdalaria, Bellemerea, Clauzadea, Farnoldia, Immersaria, Koerberiella, Pachyphysis, Porpidia, Romjulari, Lecidoma, Protoblastenia, Protomicarea, Psora, Psorula, Adelolecia, Arthrosporum, Bacidia, Bacidina, Biatora, Catinaria, Cliostomum, Desmazieria, Fistulariella, Herteliana, Japewia, Lecania, Mycobilimbia, Myxobilimbia, Niebla, Phyllopsora, Ramalina, Schadonia, Scutula, Speerschneidera, Tephromela, Trichoramalina, Waynea, Catolechia, Epilichen, Poeltinula, Rhizocarpon, Amphiloma, Hertelidea, Lepraria, Squamarina, Stereocaulon, Xyleborus, Bunodophoron, Sphaerophorus, Coccocarpia, Spilonema, Spilonemella, Collema, Leciophysma, Leptogium, Physma, Staurolemma, Degelia, Erioderma, Fuscopannaria, Leioderma, Moelleropsis, Pannaria, Parmeliella, Protopannaria, Psoroma, Santessoniella, Vahliella, Dendriscocaulon, Lobaria, Pseudocyphellaria, Sticta, Nephroma, Peltigera, Solorina, Koerberia, Leptochidium, Placynthium, Polychidium, Vestergrenopsis, Massalongia, Catillaria, Halecania, Solenopsora, Sporastatia, Toninia, Xanthopsorella, Letrouitia, Microcalicium, Caloplaca, Cephalophysis, Fulgensia, Seirophora, Teloschistes, Xanthomendoza, Xanthoria, Agyrium, Anzina, Lignoscripta, Lithographa, Placopsis, Placynthiella, Ptychographa, Rimularia, Sarea, Trapelia, Trapeliopsis, Xylographa, Anamylopsora, Hafellnera, Schaereria, Coenogonium, Belonia, Bryophagus, Cryptolechia, Gyalecta, Pachyphiale, Ramonia, Arthotheliopsis, Asterothyrium, Aulaxina, Bullatina, Calenia, Diploschistella, Echinoplaca, Gomphillus, Gyalectidium, Gyalideopsis, Jamesiella, Sagiolechia, Tricharia, Acanthothecis, Anomomorpha, Carbacanthographis, Diorygma, Dyplolabia, Fissurina, Glyphis, Graphina, Graphis, Gyrostomum, Helminthocarpon, Leiorreuma, Medusulina, Phaeographis, Platygramme, Platythecium, Sarcographa, Thalloloma, Thecaria, Geltingia, Lethariicola, Odontotrema, Skyttea, Spirographa, Thamnogalla, Xerotrema, Gyalidea, Solorinella, Absconditella, Conotrema, Nanostictis, Petractis, Robergea, Thelopsis, Topelia, Chapsa, Diploschistes, Fibrillithecis, Ingvariella, Leptotrema, Leucodecton, Melanotrema, Myriotrema, Nadvornikia, Ocellularia, Phaeotrema, Platygrapha, Reimnitzia, Stegobolus, Thelotrema, Topeliopsi, Platygraphopsis, Dibaeis, Icmadophila, Siphula, Thamnolia, Aspicilia, Lobothallia, Megaspora, Loxosporopsis, Ochrolechia, Pertusaria, Segestria, Varicellaria, Clathroporina, Porina, Pseudosagedia, Trichothelium, Arctomia, Eiglera, Hymenelia, Ionaspis, Melanolecia, Pachyospora, Tremolecia, Aspilidea, Brigantiaea, Chaenotheca, Coniocybe, Sclerophora, Fuscidea, Lettauia, Maronea, Orphniospora, Ropalospora, Phlyctella, Phlyctis, Lasallia, Umbilicaria, Vezdaea, Biatoridium, Botryolepraria, Corticifraga, Helocarpon, Leprocaulon, Malcomiella, Piccolia, Strangospora, Bryoscyphus, Unguiculariopsis, Pezizella, Llimoniella, Phaeopyxis, Phragmonaevia, Rhymbocarpus, Skyttella, Myxotrichum, Gloeoheppia, Heppia, Solorinaria, Anema, Collemopsis, Cryptothele, Digitothyrea, Ephebe, Euopsis, Harpidium, Lemmopsis, Lempholemma, Leprocollema, Lichina, Lichinella, Lichinodium, Metamelanea, Paulia, Peccania, Phloeopeccania, Phylliscum, Porocyphus, Psorotichia, Pterygiopsis, Pyrenopsis, Stromatella, Synalissa, Thelignya, Thermutis, Thyrea, Zahlbrucknerella, Peltula, Lasiosphaeriopsis, Rhagadostoma, Dendrodochium, Nectriella, Nectriopsis, Paranectria, Pronectria, Trichonectria, Nectria, Niesslia, Illosporiopsis, Illosporium, Graphium, Lichenochora, Globosphaeria, Roselliniella, Roselliniopsis, Physalospora, Obryzum, Neolamya, Sarcopyrenia, Thelidiella, Lahmia, Aspidothelium, Kohlmeyera, Mastodia, Turgidosculum, Julella, Thelenella, Baeomyces, Coccotrema, Sarcosagium, Thelocarpon, Abrothallus, Acaroconium, Bispora, Cheiromycina, Coniambigua, Dictyocatenulata, Flakea, Hawksworthiana, Heterocyphelium, Hobsoniopsis, Intralichen, Kalchbrenneriella, Kirschsteiniothelia, Lichenopuccinia, Minutoexcipula, Normandina, Patriciomyces, Phaeosporobolus, Refractohilum, Sclerococcum, Talpapellis, Tylophoron, Vouauxiomyces, Acantholichen, Cyphellostereum, Dictyonema, Lichenomphalia, Arrhenia, Fayodia, Omphalina, Semiomphalina, Athelia, Athelopsis, Lepidostroma, Leucogyrophana, Multiclavula, Marchandiomyces, Syzygospora, Tremella, Marchandiomphalina, Chionosphaera, Hobsonia, Chrysopsora, Biatoropsis, Cystobasidium, Asterophoma, Bachmanniomyces, Cornutispora, Dinemasporium, Diplolaeviopsis, Epaphroconidia, Epicladonia, Everniicola, Karsteniomyces, Laeviomyces, Lichenoconium, Lichenodiplis, Lichenosticta, Nigropuncta, Pyrenotrichum, Rhabdospora, Vouauxiella, and Xanthopsora genera.

Some more detailed examples of lichen species include Rhizocarpon advenulum, Rhizocarpon alpicola, Rhizocarpon amphibium, Rhizocarpon anaperum, Rhizocarpon arctogenum, Rhizocarpon atroflavescens, Rhizocarpon austroamphibium, Rhizocarpon badioatrum, Rhizocarpon bolanderi, Rhizocarpon caeruleoalbum, Rhizocarpon caesium, Rhizocarpon chioneum, Rhizocarpon cinereovirens, Rhizocarpon concentricum, Rhizocarpon diploschistidina, Rhizocarpon epispilum, Rhizocarpon eupetraeoides, Rhizocarpon eupetraeum, Rhizocarpon expallescens, Rhizocarpon furax, Rhizocarpon furfurosum, Rhizocarpon geminatum, Rhizocarpon geographicum, Rhizocarpon grande, Rhizocarpon hochstetteri, Rhizocarpon inarense, Rhizocarpon infernulum, Rhizocarpon intermediellum, Rhizocarpon jemtlandicum, Rhizocarpon lavatum, Rhizocarpon lecanorinum, Rhizocarpon leptolepis, Rhizocarpon macrosporum, Rhizocarpon mahreri, Rhizocarpon mosigiae, Rhizocarpon nidificum, Rhizocarpon obscuratum, Rhizocarpon oederi, Rhizocarpon oxydatum, Rhizocarpon petraeum, Rhizocarpon plicatile, Rhizocarpon polycarpum, Rhizocarpon postumum, Rhizocarpon purpurascens, Rhizocarpon rapax, Rhizocarpon reductum, Rhizocarpon renneri, Rhizocarpon richardii, Rhizocarpon ridescens, Rhizocarpon santessonii, Rhizocarpon saurinum, Rhizocarpon simillimum, Rhizocarpon sorediosum, Rhizocarpon subareolatum, Rhizocarpon subgeminatum, Rhizocarpon sublavatum, Rhizocarpon superficiale, Rhizocarpon timdalii, Rhizocarpon trapeliicola, Rhizocarpon umbilicatum, and Rhizocarpon viridiatrum.

The lichen extract is present in an amount of from about 0.1% to about 20% by weight of the total composition, or from about 0.1% to about 10%, or from about 0.1% to about 5% by weight of the total composition, or about 5% by weight of the total composition, or about 4% by weight of the total composition, or about 3% by weight of the total composition, or about 2% by weight of the total composition, or about 1% by weight of the total composition, or about 0.5% by weight of the total composition. The lichen-derived chromophore is present in an amount of from about 0.0001% to about 10% by weight of the total composition, or from about 0.0001% to about 5% by weight of the total composition, or from about 0.0001% to about 4% by weight of the total composition, or from about 0.0001% to about 3% by weight of the total composition, or from about 0.0001% to about 2% by weight of the total composition, or from about 0.0001% to about 1% by weight of the total composition, or from about 0.0001% to about 0.5% by weight of the total composition, or from about 0.0001% to about 0.25% by weight of the total composition, or from about 0.0001% to about 0.1% by weight of the total composition, or about 0.05% by weight of the total composition.

In some embodiments, the composition of the present disclosure can also include, in addition to the lichen extract(s), other cosmetic and dermatological agents, including, but not limited to: pro-collagen agents; agents which stimulate the development of the dermal/lipid layers, agents which improve firmness and elasticity, healing factors; humectants; anti-acne agents; anti-aging agents; anti-wrinkling agents, agents which reduce the appearance of fine lines, wrinkles and/or stretch marks, healing agents; deodorants and antiperspirants; skin emollients and skin moisturizers; skin lightening agents; depilating agents; counterirritants; make-up preparations; emulsifiers, vitamins; amino acids and their derivatives; herbal extracts; sensory markers (i.e., cooling agents, heating agents, of the like); skin conditioners; skin rejuvenators, chelating agents; fragrance, nourishing agents; moisture absorbers; sebum absorbers and the like; skin penetration enhancers; emollients, moisturizers and slip agents.

Additional components that may optionally be included in the composition include:

(b) Collagens and Agents that Promote Collagen Synthesis

According to some embodiments, the biophotonic compositions of the present disclosure may optionally further comprise one or more collagens and/or agents that promote collagen synthesis. Collagen is a fibrous protein produced in dermal fibroblast cells and forming 70% of the dermis. Collagen is responsible for the smoothing and firming of the skin. Therefore, when the synthesis of collagen is reduced, skin aging will occur, and so the firming and smoothing of the skin will be rapidly reduced. As a result, the skin will be flaccid and wrinkled. On the other hand, when metabolism of collagen is activated by the stimulation of collagen synthesis in the skin, the components of dermal matrices will be increased, leading to effects, such as wrinkle improvement, firmness improvement and skin strengthening. Thus, collagens and agents that promote collagen synthesis may also be useful in the present disclosure. Agents that promote collagen synthesis (i.e., pro-collagen synthesis agents) include amino acids, peptides, proteins, lipids, small chemical molecules, natural products and extracts from natural products.

For instance, it was discovered that intake of vitamin C, iron, and collagen can effectively increase the amount of collagen in skin or bone. See, e.g., U.S. Patent Application Publication 20090069217, incorporated herein by reference. Examples of the vitamin C include an ascorbic acid derivative such as L-ascorbic acid or sodium L-ascorbate, an ascorbic acid preparation obtained by coating ascorbic acid with an emulsifier or the like, and a mixture containing two or more of those vitamin Cs at an arbitrary rate. In addition, natural products containing vitamin C such as acerola and lemon may also be used. Examples of the iron preparation include: an inorganic iron such as ferrous sulfate, sodium ferrous citrate, or ferric pyrophosphate; an organic iron such as heme iron, ferritin iron, or lactoferrin iron; and a mixture containing two or more of those irons at an arbitrary rate. In addition, natural products containing iron such as spinach or liver may also be used. Moreover, examples of the collagen include: an extract obtained by treating bone, skin, or the like of a mammal such as bovine or swine with an acid or alkaline; a peptide obtained by hydrolyzing the extract with a protease such as pepsine, trypsin, or chymotrypsin; and a mixture containing two or more of those collagens at an arbitrary rate. Collagens extracted from plant sources may also be used. Creatine (such as Tego Cosmo C) and Matrixyl 3000 (Glycerin, Butylene Glycol, Carbomer, Polysorbate 20, Palmitoyl Oligopeptide, Palmitoyl Tetrapeptide-7) may also be used to stimulate dermal collagen. Additional pro-collagen synthesis agents are described, for example, in U.S. Pat. No. 7,598,291, 7,722,904, 6,203,805, 5,529,769, etc, and U.S. Patent Application Publications 20060247313, 20080108681, 20110130459, 20090325885, 20110086060, etc., the contents of all of which are incorporated herein by reference.

(c) Carrier Medium

In some embodiments, the biophotonic compositions of the present disclosure comprise a carrier medium made from one or more thickening agents. Thickening agents are present in an amount and ratio sufficient to provide a desired viscosity, flexibility, rigidity, tensile strength, tear strength, elasticity, and adhesiveness. The thickening agents are selected so that the chromophore(s) of the lichen extract(s) can remain photoactive in the carrier medium. The thickening agents are also selected according to the optical transparency of the carrier medium. The carrier medium should be able to transmit sufficient light to activate the at least one chromophore and, in embodiments where fluorescence is emitted by the activated chromophore, the carrier medium should also be able to transmit the emitted fluorescent light to tissues. It will be recognized by persons skilled in the art that the thickening agent is an appropriate medium for lichen extract or the lichen-derived chromophores. For example, for xanthene dyes that do not fluoresce in non-hydrated media, hydrated polymers or polar solvents may be used. The thickening agents should also be selected according to the intended use. For example, if the biophotonic composition is to be applied onto tissue, the carrier medium is preferably biocompatible, or the carrier medium has an outside layer of a biocompatible composition which will interface the tissue.

Thickening Agents

According to some embodiments, the biophotonic compositions of the present disclosure may optionally further comprise at least one thickening agent. In some embodiments, the content of a thickening agent is present in the composition in an amount of from about 0.001% to about 40% (w/w %) of the total weight. In certain embodiments, the total content of the thickening agent is about 0.001%-0.01%, about 0.005%-0.05%, about 0.01%-0.1%, about 0.05%-0.5%, about 0.1%-1%, about 0.5%-5%, about 1%-5%, about 2.5%-7.5%, about 5%-10%, about 7.5%-12.5%, about 10%-15%, about 12.5%-17.5%, about 15%-20%, about 15%-25%, about 20%-30%, about 25%-35%, or about 30%-40% by weight of the total composition. In some embodiments, the total content of the thickening agent is 0.001%-0.01%, 0.005%-0.05%, 0.01%-0.1%, 0.05%-0.5%, 0.1%-1%, 0.5%-5%, 1%-5%, 2.5%-7.5%, 5%-10%, 7.5%-12.5%, 10%-15%, 12.5%-17.5%, 15%-20%, 15%-25%, 20%-30%, 25%-35%, or 30%-40% by weight of the total composition. It will be recognized by one of skill in the art that the viscosity, flexibility, rigidity, tensile strength, tear strength, elasticity, and adhesiveness can be adjusted by varying the content of the thickening agent. Methods of determining viscosity, flexibility, rigidity, tensile strength, tear strength, elasticity, and adhesiveness are known in the art.

Thickening agents that can be used to prepare the biophotonic compositions of the present disclosure include but are not limited to a hydrophilic polymer, a hygroscopic polymer or a hydrated polymer. The thickening agent may be polyanionic in charge character. The thickening agent may comprise carboxylic functional groups, and may further contain 2 to 7 carbon atoms per functional group. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, N,N-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hyaluronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propylsulfonizacid-3)ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In some embodiments, the thickening agent comprises 2-Hydroxyethyl methacrylate (HEMA) either alone or in addition to another thickening agent. In some embodiments, the 2-Hydroxyethyl methacrylate (HEMA) is added in a form such as microspheres or in a further physically reduced form such as a finely ground particulate form or in a pulverized, powder form.

In certain embodiments, the at least one thickening agent is a synthetic polymer selected from one or more of vinyl polymers, polyoxythylene-polyoxypropylene copolymers, poly(ethylene oxide), acrylamide polymers and derivatives and salts thereof. In a further embodiment, the vinyl polymer is selected from one or more of polyacrylic acid, polymethacrylic acid, polyvinyl pyrrolidone, and polyvinyl alcohol. In other embodiments, the vinyl polymer is a carboxy vinyl polymer or a carbomer obtained by the polymerization of acrylic acid. The carboxy vinyl polymer or carbomer may be cross-linked.

As mentioned above, in some embodiments, the at least one thickening agent of the carrier medium comprises one or more carbomers. Carbomers are synthetic high molecular weight polymers of acrylic acid that are crosslinked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomers are available as fine white powders which disperse in water to form acidic colloidal suspensions (a 1% dispersion has approximately pH 3) of low viscosity. Neutralization of these suspensions using a base, for example sodium, potassium or ammonium hydroxides, low molecular weight amines and alkanolamines, results in the formation of clear translucent gels.

In some embodiments, the carbomer is a Carbopol®. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like. Carbopols are versatile controlled-release polymers, as described by Brock (Pharmacotherapy, 14:430-7 (1994), incorporated herein by reference) and Durrani (Pharmaceutical Res. (Supp.) 8:S-135 (1991), incorporated herein by reference), and belong to a family of carbomers which are synthetic, high molecular weight, non-linear polymers of acrylic acid, cross-linked with polyalkenyl polyether. In certain embodiments, the carbomer is Carbopol® 940, Carbopol® 980, ETD 2020NF, Carbopol® 1382, 71G NF, 971P NF, 974P NF, 980 NF, 981 NF, 5984 EP, ETF 2020 NF, Ultrez 10 NF, Ultrez 20, Ultrez 21, 1342 NF, 934 NF, 934P NF, 940 NF or 941 NF, or combinations thereof. In some embodiments, the carbomer is cross-linked with alkyl acrylate or allyl pentaerythritol. In some embodiments, the carbomer is present in the composition in an amount of from about 0.01 wt % to about 15 wt %, or about 0.05 wt % to about 5 wt %, or about 0.5 wt % to about 2 wt %. In some embodiments, the carbomer is present in the composition in an amount of from 0.01 wt % to 15 wt %, or 0.05 wt % to 5 wt %, or 0.5 wt % to 2 wt %.

In certain embodiments, the at least one thickening agent of the carrier medium is a glycol, such as ethylene glycol or propylene glycol. In further embodiments, the at least one thickening agent of the carrier medium is a poly(ethylene oxide) polymer (such as POLYOX from Dow Chemical), linear PVP and cross-linked PVP, PEG/PPG copolymers (such as BASF Pluracare L1220), ethylene oxide (EO)-propylene oxide (PO) block copolymers (such as polymers sold under the trade mark Pluronic available from BASF Corporation), ester gum, shellac, pressure sensitive silicone adhesives (such as BioPSA from Dow-Corning), or mixtures thereof. In some embodiments, the at least one thickening agent of the carrier medium is a copolymer. In certain such embodiments, the copolymer comprises (PVM/MA). In some embodiments, the copolymer comprises poly(methylvinylether/maleic anhydride). In some embodiments, the copolymer comprises poly (methylvinylether/maleic acid). In some embodiments, the copolymer comprises poly(methylvinylether/maleic acid) half esters. In some embodiments, the copolymer comprises poly(methylvinylether/maleic acid) mixed salts.

In certain embodiments of the disclosure, the at least one thickening agent of the carrier medium is a protein-based polymer. In certain such embodiments, the protein-based polymer may be selected from at least one of elastin, gelatin, or collagen. For example, the composition may comprise at least about 4 wt %, about 4 wt % to about 25 wt %, or about 10 wt % to about 20 wt % gelatin within the biophotonic composition. In some embodiments, the composition may comprise at least 4 wt %, 4 wt % to 25 wt %, or 10 wt % to 20 wt % gelatin within the biophotonic composition. The composition may comprise at least about 5 wt %, about 5 wt % to about 25 wt %, or about 10 wt % to about 20 wt % collagen and/or elastin within the biophotonic composition. In some embodiments, the composition may comprise at least 5 wt %, 5 wt % to 25 wt %, or 10 wt % to 20 wt % collagen and/or elastin within the biophotonic composition. Alternatively, a lower weight percentage of protein-based polymers may be used together with chemical cross-linkers or any other cross-linking means.

In certain embodiments of the disclosure, the at least one thickening agent of the carrier medium is a polysaccharide selected from the group consisting of sodium hyaluronate, starch, chitosan, chitin, agar, alginates, xanthan, carrageenan, guar gum, gellan gum, pectin, locust bean gum, hydroxylpropyl cellulose, carboxymethyl cellulose, and combinations thereof.

(d) Healing Factors

In some embodiments, the biophotonic compositions of the present disclosure comprise at least one healing factor. Healing factors comprise compounds that promote or enhance the healing or regenerative process of the tissues on the application site of the composition. In certain embodiments, during the photoactivation of the composition of the present disclosure, an increase of the absorption of molecules at the treatment site by the skin or the mucosa is observed. In certain embodiments, an augmentation in the blood flow at the site of treatment is also observed over an extended period of time. An increase in the lymphatic drainage and a possible change in the osmotic equilibrium due to the dynamic interaction of the free radical cascades can be enhanced or even fortified with the inclusion of healing factors.

Suitable healing factors include, but are not limited to, hyaluronic acid, glucosamine, and allantoin.

Hyaluronic acid (Hyaluronan, hyaluronate) is a non-sulfated glycosaminoglycan, distributed widely throughout connective, epithelial and neural tissues. It is one of the primary components of the extracellular matrix, and contributes significantly to cell proliferation and migration. Hyaluronan is a major component of the skin, where it is involved in tissue repair. While it is abundant in extracellular matrices, it contributes to tissues hydrodynamics, movement and proliferation of cells and participates in a wide number of cell surface receptor interactions, notably those including primary receptor CD44. The hyaluronidases enzymes degrade hyaluronan. There are at least seven types of hyaluronidase-like enzymes in humans, several of which are tumor suppressors. The degradation products of hyaluronic acid, the oligosaccharides and the very-low molecular weight hyaluronic acid, exhibit pro-angiogenic properties. In addition, recent studies show that hyaluronan fragments, but not the native high molecular mass of hyaluronan, can induce inflammatory responses in macrophages and dendritic cells in tissue injury. Hyaluronic acid is well suited to biological applications targeting the skin. Due to its high biocompatibility, it is used to stimulate tissue regeneration. Current studies evidenced hyaluronic acid appearing in the early stages of healing to physically create room for white blood cells that mediate the immune response. It is used in the synthesis of biological scaffolds for wound healing applications and in wrinkle treatment.

Glucosamine is one of the most abundant monosaccharides in human tissues and a precursor in the biological synthesis of glycosylated proteins and lipids. It is commonly used in the treatment of osteoarthritis. The common form of glucosamine used is its sulfate salt. Glucosamine shows a number of effects including an anti-inflammatory activity, stimulation of the synthesis of proteoglycans and the synthesis of proteolytic enzymes. A suitable range of concentration over which glucosamine can be used in the present composition is from about 1% to about 3%. Another form of glucosamine which can be used is acetyl glucosamine.

Allantoin is a diureide of glyosilic acid. It has keratolytic effect, increases the water content of the extracellular matrix, enhances the desquamation of the upper layers of dead (apoptotic) skin cells, and promotes skin proliferation and wound healing. It is considered as an anti-irritant. Also, saffron can act as both a chromophore and a healing factor.

(e) Vitamins

In certain embodiments, the compositions of the present disclosure further comprise one or more vitamins. For example, vitamin A and derivatives thereof, vitamin B2, biotin, pantothenic acid, vitamin K, vitamin D, vitamin E, vitamin C, and mixtures thereof can be used.

(f) Skin Barrier Repair Actives

According to some embodiments, the biophotonic compositions the present disclosure may optionally further comprise one or more skin barrier repair actives. Skin barrier repair actives are those skin care actives which can help repair and replenish the natural moisture barrier function of the epidermis and can be included in the biophotonic composition. Non-limiting examples of skin barrier repair actives include Alpha Lipid (available from Lucas Meyer); ascorbic acid; biotin; biotin esters; brassicasterol; caffeine; campesterol; canola derived sterols; Cennamides (available from Ennagram); Ceramax (available from Alban Muller); CERAMAX (available from Quest, located in Ashford, England); CERAMIDE 2 and CERAMIDE HO3 (both available from Sederma); CERAMIDE II (available from Quest); CERAMIDE III and IIIB (both available from Cosmoferm, located in Deft, Netherlands); CERAMIDE LS 3773 (available from Laboratories Serobiologiques); CERAMINOL (available from Inocosm); Cerasol and Cephalip (both available from Pentapharm); cholesterol; cholesterol hydroxystearate; cholesterol isostearate; 7 dehydrocholesterol; DERMATEIN BRC and DERMATEIN GSL (both available from Hormel); ELDEW CL 301 AND ELDEW PS 203 (both available from Ajinomoto); Fitobroside (available from Pentapharm); galactocerebrosides; Generol 122 (available from Henkel); glyceryl serine amide; hydroxyethyl isostearyl isopropanolamine; lactic acid; Lactomide (available from Pentapharm); lanolin; lanolin alcohols; lanosterol; lauric acid N laurylglucamide; lipoic acid; N-acetyl cysteine; N-acetyl-L-serine; N-methyl-L-Serine; Net Sterol-ISO (available from Barnet Products); vitamin B3 compounds (such as niacinamide and nicotinic acid); palmitic acid; panthenol; panthetine; phosphodiesterase inhibitors; PHYTO/CER (available from Intergen); phytoglycolipid millet extract (available from Barnet Products Distributer, located in Englewood, N.J.); PHYTOSPHINGOSINE (available from Gist Brocades, located in King of Prussia, Pa.); PSENDOFILAGGRIN (available from Brooks Industries, located in South Plainfield, N.J.); QUESTAMIDE H (available from Quest); serine; sigmasterol; sitosterol; soybean derived sterols; sphingosine; sphingomylinase; S-lactoyl glutathione; stearic acid; Structurine (available from Silah); SUPER STEROL ESTERS (available from Croda); thioctic acid; THSC CERAMIDE OIL (available from Campo Research); trimethyl glycine; tocopheryl nicotinate; vitamin D3; Y2 (available from Ocean Pharmaceutical); and mixtures thereof,

(g) Non-Steroidal Cosmetic Soothing Actives

According to some embodiments, the biophotonic compositions of the present disclosure may optionally further comprise one or more cosmetic soothing actives. Cosmetic soothing actives can be effective in preventing or treating inflammation of the skin and can be included in composition of the present disclosure. The soothing active enhances the skin appearance benefits of the provided by the present composition, e.g., such agents contribute to a more uniform and acceptable skin tone or color. The exact amount of anti-inflammatory agent to be used in the compositions will depend on the particular anti-inflammatory agent utilized since such agents vary widely in potency. Non-limiting examples of cosmetic soothing agents include the following categories: propionic acid derivatives; acetic acid derivatives; fenamic acid derivatives; biphenylcarboxylic acid derivatives; and oxicams. Non-limiting examples of useful cosmetic soothing actives include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, absinthium, acacia, aescin, alder buckthorn extract, allantoin, aloe, APT (available from Centerchem), arnica, astragalus, astragalus root extract, azulene, Baicalin SR 15 (available from Bamet Products Dist.), baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (available from Fytokem), candelilla wax, Cangzhu, canola phytosterols, capsicum, carboxypeptidase, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GLUCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; etbacrynic acid, evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), Emulium (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and mixtures thereof.

(h) Skin Conditioners

According to some embodiments, the biophotonic compositions of the present disclosure may optionally further comprise one or more skin conditioners, moisturizers and surfactants as additives. Illustrative conditioners include mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are Propane-1,3-diol, polyols such as sorbitol, glycerin, propylene glycol, ethylene glycol, polyethylene glycol, polypropylene glycol, 1,3-butane diol, hexylene glycol, isoprene glycol, xylitol, fructose, Aqualance (Erythritol, Homarine, HCl), antarcticine with salicyclic acid (Pseudoalteromonas ferment Extract, Salicyclic acid, sodium hydroxide), Cristalhyal FL (Sodium Hyaluronate, 1,2-hexanediol 9, caprylyl glycol), Iricalmin (Triticum Vulgare (wheat) germ extract, saccharomyces Cerevisiae, Sodium Hyaluronate, Panthenol), Nibi (Sambucus Nigra Flower extract), and mixtures thereof.

Preservatives

According to some embodiments, the biophotonic compositions of the present disclosure may optionally further comprise one or more preservatives. Preservatives can desirably be incorporated into the composition of the present disclosure. Suitable preservatives for compositions of the present disclosure include but are not limited to alkyl esters of parahydroxybenzoic acid. Other preservatives, which can be used include hydantoin derivatives, propionate salts, and a variety of quaternary ammonium compounds. Appropriate preservatives can be selected to satisfy the preservative challenge test and to provide product stability. Particularly preferred preservatives are phenoxyethanol, methylparaben, imidazolidinyl urea, sodium dehydroacetate, propylparaben, trisodium ethylenediamine tetraacetate (EDTA), benzyl alcohol, phenoxyethanol and ethylhexyglycerin. The preservative can be selected based on the consideration of possible incompatibilities between the preservative and other ingredients in the release system.

The compositions of the disclosure may also include gelling agents. Suitable gelling agents for aqueous gels include, but are not limited to, natural gums, polysaccharides, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Starches may also be used as the gelling agent.

(j) Chelating Agents

In some embodiments, the biophotonic compositions of the present disclosure further comprise one or more chelating agent. A chelating agent can be included to promote smear layer removal in closed pockets and difficult to reach lesions. Chelating agents act as a metal ion quencher and as a buffer. Suitable chelating agents for the compositions of the disclosure include, but are not limited to:

Ethylenediaminotetraacetic Acid (EDTA)

Ethylenediaminotetraacetic acid (EDTA) is an amino acid and is used to sequester di- and trivalent metal ions. EDTA binds to metals via four carboxylate and two amine groups. EDTA forms especially strong complexes with Mn(III), Fe(III), Cu(III), Co(III). It is used to buffer solutions.

Ethylene Glycol Tetraacetic Acid (EGTA)

Ethylene glycol tetraacetic acid (EGTA) is related to EDTA, but with a much higher affinity for calcium than magnesium ions. It is useful for making buffer solutions that resemble the environment inside living cells.

(k) Additional Components

The compositions of the disclosure can also include other ingredients such as humectants (e.g., glycerine, ethylene glycol, and propylene glycol), preservatives such as parabens, and pH adjusters such as sodium hydroxide, sodium bicarbonate, and HCl. In some embodiments, the pH of the composition is in or adjusted to the range of about 4 to about 10. In some embodiments, the pH of the composition is in or adjusted to the range of about 4 to about 9, such as about 4 to about 8, about 4 to about 7, about 4 to about 6.5, about 4 to about 6, about 4 to about 5.5, or about 4 to about 5. In some embodiments, the pH of the composition is within the range of about 5.0 to about 8.0. In some embodiments, the pH of the composition is within the range of about 6.0 to about 8.0, such as about 6.5 to about 7.5. In some embodiments, the pH of the composition is within the range of about 5.5 to about 7.5, such as about 5.5.

In some embodiments, the pH of the composition is in or adjusted to the range of 4 to 10. In some embodiments, the pH of the composition is in or adjusted to the range of 4 to 9, such as 4 to 8, such as 4 to 7, such as 4 to 6.5, such as 4 to 6, such as 4 to 5.5. In some embodiments, the pH of the composition is within the range of 4 to 5. In some embodiments, the pH of the composition is within the range of 5.0 to 8.0. In some embodiments, the pH of the composition is within the range of 6.0 to 8.0. In some embodiments, the pH of the composition is within the range of 6.5 to 7.5. In some embodiments, the pH of the composition is within the range of 5.5 to 7.5, such as 5.5.

In some embodiments, the compositions of the disclosure also include an aqueous substance (e.g., water or purified water) or an alcohol. Alcohols include, but are not limited to, ethanol, propanol, isopropanol, butanol, iso-butanol, t-butanol or pentanol. In some embodiments, the lichen extract or combination of lichen extracts is in a medium or in a solution of the biophotonic composition. In certain such embodiments, the medium is an aqueous substance. In some embodiments, the lichen-derived chromophore or combination of lichen derived chromophores is in a medium or a solution of the biophotonic composition. In certain such embodiments, the medium is an aqueous substance.

Certain suitable compositions of the disclosure can be described based on the absence of certain components from the composition. The examples provided herein may be combined so that a suitable composition may specifically exclude one or more of these ingredients. For example, in certain embodiments, the composition does not include polyphenols which can filter UV light. In certain embodiments, the composition does not include self-tanning agents. In certain embodiments, the concentration of the lichen extracts in the composition is not so high so as to give the composition an unnatural or non-neutral coloring. In certain embodiments, the concentration of the lichen extracts is not so high so that the lichen extracts can be activated by low intensity light, such as ambient light or a direct light. In certain embodiments, the concentration of the lichen-derived chromophore(s) in the composition is not so high so as to give the composition an unnatural or non-neutral coloring. In certain embodiments, the concentration of the lichen derived chromophore(s) is not so high so that the lichen-derived chromophore(s) can be activated by low intensity light, such as ambient light or a direct light.

### (4) Light Source

The present disclosure provides methods of treating skin by topically applying the compositions of the present disclosure and further illuminating with a light source, e.g., a phototherapy device or a lighting device, such as in the form of a mask or a portable hand-held foldable panels. In certain embodiments, the lighting device comprises a plurality of light sources, e.g., LEDs that are disposed in an assembly inside a hands free mask to apply light therapy to the area of treatment. The light source or means of light emission comprises pulsed light; focused light (e.g., lasers, such as low energy lasers), diffuse, multi wavelength, single wavelength, visible and/or non-visible light wavelengths.

In the methods of the present disclosure, any source of actinic light can be used. The source of actinic light may be a natural source, such as sunlight, or may be a generated source. Any type of halogen, LED or plasma arc lamp or laser may be suitable source of generated actinic light. The primary characteristic of suitable sources of actinic light will be that they emit light in a wavelength (or wavelengths) appropriate for activating the one or more photoactivators present in the composition. The appropriate wavelength (or wavelengths) may be in the visible range of wavelengths of light, or may be of a shorter wavelength or of a longer wavelength (e.g., infra red) than visible light. In some embodiments, an argon laser is used. In other embodiments, a potassium-titanyl phosphate (KTP) laser (e.g., a GreenLight™ laser) is used. In yet other embodiments, a LED lamp such as a photocuring device is the source of the actinic light.

In some embodiments, the source of the actinic light is a source of light having a wavelength from about 380 to about 800 nm. In certain such embodiments, the source of the actinic light is a source of light having a peak wavelength from about 600-700 nm, such as a maximum peak wavelength of 640 nm and a minimum peak wavelength of 620 nm. In other such embodiments, the source of the actinic light is a source of light having a peak wavelength from about 380-500 nm, such as a maximum peak wavelength of 450 nm and a minimum peak wavelength of 425 nm. In other embodiments, the source of the actinic light is a source of visible light having a wavelength from about 400 nm and about 600 nm. In other embodiments, the source of the actinic light is a source of visible light having a wavelength from about 400 nm and about 700 nm or about 400 nm to about 750 nm. In certain embodiments, suitable peak power of the LEDs are in the range from about 100 μW to 1000 μW, from about 200 μW to 900 μW, from 300 μW to 800 μW, or about 300 μW, or about 400 μW, or about 500 μW, or about 600 μW, or about 700 μW, or about 800 μW, or about 900 μW.

In yet other embodiments, the source of the actinic light is blue light. In yet other embodiments, the source of the actinic light is red light. In yet other embodiments, the source of the actinic light is green light. In some embodiments, the LED lamp may comprise LEDs of more than one wavelength, for example, LEDs that emit at a blue light range and other LEDs that emit at the green light or yellow light range or red light range or other ranges of light. Furthermore, the source of actinic light should have a suitable power density. Suitable power densities for non-collimated light sources (LED, halogen or plasma lamps) are in the range from about 0.1 mW/cm² to about 200 mW/cm², or about 30 mW/cm² to about 150 mW/cm². Suitable power densities for laser light sources are in the range from about 0.5 mW/cm² to about 0.8 mW/cm².

In some embodiments, the light has an energy at the subject's skin surface of between about 0.1 mW/cm² and about 500 mW/cm², or between about 0.1 mW/cm² and about 300 mW/cm², or between about 0.1 mW/cm² and about 200 mW/cm², wherein the energy applied depends at least on the condition being treated, the wavelength of the light, the distance of the skin from the light source and the thickness of the biophotonic composition. In certain embodiments, the light at the subject's skin is between about 0.1 mW/cm² and about 0.5 mW/cm², or between about 0.1 mW/cm² and about 0.6 mW/cm², or between about 0.1 mW/cm² and about 0.7 mW/cm², or between about 0.1 mW/cm² and about 0.8 mW/cm², or between about 0.1 mW/cm² and about 0.9 mW/cm², or between about 0.1 mW/cm² and about 1 mW/cm², or between about 1 mW/cm² and about 40 mW/cm², or between about 20 mW/cm² and about 60 mW/cm², or between about 40 mW/cm² and about 80 mW/cm², or between about 60 mW/cm² and about 100 mW/cm², or between about 80 mW/cm² and about 120 mW/cm², or between about 100 mW/cm² and about 140 mW/cm², or between about 30 mW/cm² and about 180 mW/cm², or between about 120 mW/cm² and about 160 mW/cm², or between about 140 mW/cm² and about 180 mW/cm², or between about 160 mW/cm² and about 200 mW/cm², or between about 110 mW/cm² and about 240 mW/cm², or between about 110 mW/cm² and about 150 mW/cm², or between about 190 mW/cm² and about 240 mW/cm².

The activation of the chromophore(s) within the biophotonic compositions of the disclosure may take place almost immediately upon illumination (e.g., within femto- or pico seconds). A prolonged exposure period may be beneficial to exploit the synergistic effects of the absorbed, reflected and reemitted light of the biophotonic compositions of the present disclosure and its interaction with the tissue being treated. In some embodiments, the time of exposure to actinic light of the tissue or skin or biophotonic composition is a period between about 1 minute and about 15 minutes, such as between about 1 minute and about 10 minutes, between about 1 minute and about 5 minutes, between about 1 minute and about 3 minutes. In certain embodiments, light is applied for a period of between about 1 second and about 30 seconds, between about 15 seconds and about 45 seconds, between about 30 seconds and 60 seconds, between about 0.75 minute and about 1.5 minutes, between about 1 minute and about 2 minutes, between about 1.5 minute and about 2.5 minutes, between about 2 minutes and about 3 minutes, between about 2.5 minutes and about 3.5 minutes, between about 3 minutes and about 4 minutes, between about 3.5 minutes and about 4.5 minutes, between about 4 minutes and about 5 minutes, between about 5 minutes and about 10 minutes, between about 5 minutes and about 9 minutes, between about 5 minutes and about 8 minutes, between about 10 minutes and about 15 minutes, between about 15 minutes and about 20 minutes, between about 20 minutes and about 25 minutes, or between about 20 minutes and about 30 minutes. In some embodiments, light is applied for a period of about 5 minutes. In some embodiments, light is applied for a period of about 10 minutes. In some embodiments, light is applied for a period of about 15 minutes. In some embodiments, light is applied for a period of about 1 second. In some embodiments, light is applied for a period of about 5 seconds. In some embodiments, light is applied for a period of about 10 seconds. In some embodiments, light is applied for a period of about 20 seconds. In some embodiments, light is applied for a period of about 30 seconds. In some embodiments, the biophotonic composition is illuminated for a period less than 30 minutes, such as for a period less than 20 minutes, for a period less than 15 minutes, for a period less than 10 minutes, for a period less than 5 minutes. In some embodiments, the biophotonic composition is illuminated for a period less than 1 minute, such as for a period less than 30 seconds, for a period less than 20 seconds, for a period less than 10 seconds, for a period less than 5 seconds, for a period less than 1 second. The treatment time may range up to about 90 minutes, about 80 minutes, about 70 minutes, about 60 minutes, about 50 minutes, about 40 minutes, about 30 minutes, or about 20 minutes. It will be appreciated that the treatment time can be adjusted in order to maintain a dosage by adjusting the rate of fluence delivered to a treatment area. For example, the delivered fluence may be between about 0.01 J/cm² and about 4 J/cm², such as between about 0.01 J/cm² and about 0.5 J/cm², between about 0.01 J/cm² and about 1 J/cm², between about 1 J/cm² and about 2 J/cm², between about 2 J/cm² and about 3 J/cm², between about 3 J/cm² and about 4 J/cm² or between about 4 J/cm² and about 60 J/cm², between about 10 J/cm² and about 60 J/cm², between about 10 J/cm² and about 50 J/cm², between about 10 J/cm² and about 40 J/cm², between about 10 J/cm² and about 30 J/cm², between about 20 J/cm² and about 40 J/cm², between about 15 J/cm² and 25 J/cm², or between about 10 J/cm² and about 20 J/cm². The delivery fluence is also adjustable.

In certain embodiments, the biophotonic compositions of the disclosure may be re-illuminated at certain intervals, such as 1, 2, 3, 4, 5, 6, 7, 8, 9, 10, 11, 12, 24, 36, or 48 hours following initial illumination. In yet other embodiments, the source of actinic light is in continuous motion over the treated area for the appropriate time of exposure. In yet other embodiments, the biophotonic compositions of the disclosure may be illuminated until the biophotonic composition is at least partially photobleached or fully photobleached.

In the methods and uses of the present disclosure, the biophotonic compositions may be removed from the skin following application of light. In some embodiments, the biophotonic composition is peeled off, or is washed off, the tissue being treated after a treatment time. In other embodiments, the biophotonic composition is left on the tissue. In other embodiments, the biophotonic composition is left on for an extended period of time and re-activated with direct or ambient light at appropriate times to treat the condition.

In certain embodiments of the methods and uses of the present disclosure, the biophotonic compositions can be applied to the tissue, such as on the face or afflicted site, once, twice, three times, four times, five times or six times a week, daily, or at any other frequency. The total treatment time can be one week, two weeks, three weeks, four weeks, five weeks, six weeks, seven weeks, eight weeks, nine weeks, ten weeks, eleven weeks, twelve weeks, or any other length of time deemed appropriate. In certain embodiments, the total tissue area to be treated may be split into separate areas (cheeks, forehead), wherein each area is treated separately. For example, the biophotonic composition may be applied topically to a first portion, and that portion illuminated with light, and the biophotonic composition then removed. Then the biophotonic composition is applied to a second portion, illuminated and removed. Finally, the biophotonic composition is applied to a third portion, illuminated and removed.

### (5) Optical Properties of the Biophotonic Compositions

In certain embodiments, biophotonic compositions of the present disclosure are substantially transparent or translucent. The % transmittance of the biophotonic composition can be measured in the range of wavelengths from 250 nm to 800 nm using, for example, a Perkin-Elmer Lambda 9500 series UV-visible spectrophotometer. In some embodiments, transmittance within the visible range is measured and averaged. In some other embodiments, transmittance of the biophotonic composition is measured with the chromophore(s) omitted. As transmittance is dependent upon thickness, the thickness of each sample can be measured with calipers prior to loading in the spectrophotometer. Transmittance values can be normalized according to:

$${F}_{T - corr} ( λ , {t}_{2} ) = {[ {e}^{- {σ}_{t}} ( λ ) {t}_{1} ]}^{\frac{{t}_{2}}{{t}_{1}}} = {[ {F}_{T - corr} ( λ , {t}_{1} ) ]}^{\frac{{t}_{2}}{{t}_{1}}} ,$$

where t₁=actual specimen thickness, t₂=thickness to which transmittance measurements can be normalized. In the art, transmittance measurements are usually normalized to 1 cm.

In certain embodiments, the biophotonic compositions are substantially opaque. In these embodiments, the biophotonic compositions may include light transmitting structures such as fibers, particles, networks, which are made of materials which can transmit light. The light transmitting structures can be waveguides such as optical fibers.

In some embodiments, the biophotonic composition has a transmittance that is more than about 20%, about 25%, about 30%, about 35%, about 40%, about 45%, about 50%, about 55%, about 60%, about 65%, about 70%, or about 75% within the visible range. In some embodiments, the transmittance exceeds 40%, 41%, 42%, 43%, 44%, or 45% within the visible range.

### (6) Forms of the Biophotonic Compositions

The biophotonic compositions of the present disclosure may be a liquid, a gel, a cream, a paste, a putty, a semi-solid, a serum, a solution, a lotion, or a solid, or a clear colorless solution. Biophotonic compositions in the liquid, gel, cream, paste or putty form can be applied by spreading, spraying, smearing, dabbing or rolling the composition on the target tissue. Biophotonic compositions of the putty, semi-solid or solid forms may be deformable. They may be elastic or non-elastic (i.e., flexible or rigid). The biophotonic compositions, for example, may be in a peel-off form (‘peelable’) to provide ease and speed of use. In certain embodiments, the tear strength and/or tensile strength of the peel-off form is greater than its adhesion strength. This may help handleability of the composition. It will be recognized by one of skill in the art that the properties of the peel-off biophotonic composition such as cohesiveness, flexibility, elasticity, tensile strength, and tearing strength, can be determined and/or adjusted by methods known in the art such as by selecting suitable thickening agents and adapting their relative ratios.

The biophotonic composition may be in a pre-formed shape. In certain embodiments, the pre-formed shape is in the form of, including, but not limited to, a film, a face mask, a patch, a dressing, or bandage. The biophotonic composition can be configured with a shape and/or size for application to a desired portion of a subject's body. For example, the biophotonic composition can be shaped and sized to correspond with a desired portion of the body to receive the biophotonic treatment. Such a desired portion of the body can be selected from, but not limited to, the group consisting of a skin, head, forehead, scalp, nose, cheeks, lips, ears, face, neck, shoulder, arm pit, arm, elbow, hand, finger, abdomen, chest, stomach, back, buttocks, sacrum, genitals, legs, knee, feet, toes, nails, hair, soft tissues, any boney prominences, and combinations thereof, and the like. The biophotonic composition may also be configured to be applied internally to a subject's body, such as on the luminal surface of a body cavity or organ of a subject, or be configured to be fitted or juxtapositioned to cover a substantial portion of the subject's external body surface or surface of a limb or other extremity. Thus, the biophotonic composition of the disclosure can be shaped and sized to be applied to any portion of tissue on a subject's body. For example, the biophotonic composition can be provided in the form of sock, hat, glove or mitten.

In certain aspects, the biophotonic composition forms part of a composite and can include fibers, particulates, non-biophotonic layers or biophotonic layers with the same or different compositions.

The biophotonic compositions of the present disclosure may have a thickness of, or be applied with a thickness of less than about 0.1 mm, or from about 0.1 mm to about 50 mm, about 0.5 mm to about 20 mm, or about 1 mm to about 10 mm. It will be appreciated that the thickness of the biophotonic compositions will vary based on the intended use. In some embodiments, the biophotonic composition has a thickness of less than about 0.1 mm or from about 0.1 mm to 1 mm. In some embodiments, the biophotonic composition has a thickness of between about 0.5 mm and about 1.5 mm, between about 1 mm and about 2 mm, between about 1.5 mm and about 2.5 mm, between about 2 mm and about 3 mm, between about 2.5 mm and about 3.5 mm, between about 3 mm and about 4 mm, between about 3.5 mm and about 4.5 mm, between about 4 mm and about 5 mm, between about 4.5 mm and about 5.5 mm, between about 5 mm and about 6 mm, between about 5.5 mm and about 6.5 mm, between about 6 mm and about 7 mm, between about 6.5 mm and about 7.5 mm, between about 7 mm and about 8 mm, between about 7.5 mm and about 8.5 mm, between about 8 mm and about 9 mm, between about 8.5 mm and about 9.5 mm, between about 9 mm and about 10 mm, between about 10 mm and about 11 mm, between about 11 mm and about 12 mm, between about 12 mm and about 13 mm, between about 13 mm and about 14 mm, between about 14 mm and about 15 mm, about 15 mm and about 16 mm, between about 16 mm and about 17 mm, between about 17 mm and about 18 mm, between about 18 mm and about 19 mm, between about 19 mm and about 20 mm, between about 20 mm and about 22 mm, between about 22 mm and about 24 mm, between about 24 mm and about 26 mm, between about 26 mm and about 28 mm, between about 28 mm and about 30 mm, between about 30 mm and about 35 mm, between about 35 mm and about 40 mm, between about 40 mm and about 45 mm, or between about 45 mm and about 50 mm. In some embodiments, the biophotonic composition has a thickness of less than 0.1 mm, from 0.1 mm to 50 mm, 0.5 mm to 20 mm, or 1 mm to 10 mm. In some embodiments, the biophotonic composition has a thickness of from less than 0.1 mm, from 0.1 mm-1 mm. In some embodiments, the biophotonic composition has a thickness of between about 0.5 mm and about 1.5 mm, between about 1 mm and about 2 mm, between about 1.5 mm and about 2.5 mm, between about 2 mm and about 3 mm, between about 2.5 mm and about 3.5 mm, between about 3 mm and about 4 mm, between about 3.5 mm and about 4.5 mm, between about 4 mm and about 5 mm, between about 4.5 mm and about 5.5 mm, between about 5 mm and about 6 mm, between about 5.5 mm and about 6.5 mm, between about 6 mm and about 7 mm, between about 6.5 mm and about 7.5 mm, between about 7 mm and about 8 mm, between about 7.5 mm and about 8.5 mm, between about 8 mm and about 9 mm, between about 8.5 mm and about 9.5 mm, between about 9 mm and about 10 mm, between about 10 mm and about 11 mm, between about 11 mm and about 12 mm, between about 12 mm and about 13 mm, between about 13 mm and about 14 mm, between about 14 mm and about 15 mm, between about 15 mm and about 16 mm, between about 16 mm and about 17 mm, between about 17 mm and about 18 mm, between about 18 mm and about 19 mm, between about 19 mm and about 20 mm, between about 20 mm and about 22 mm, between about 22 mm and about 24 mm, between about 24 mm and about 26 mm, between about 26 mm and about 28 mm, between about 28 mm and about 30 mm, between about 30 mm and about 35 mm, between about 35 mm and about 40 mm, between about 40 mm and about 45 mm, or between about 45 mm and about 50 mm.

### (7) Methods of Use

The present disclosure also includes methods and uses of any of the biophotonic compositions described herein or combinations thereof. The present disclosure includes methods and uses of the biophotonic composition for treating a skin disorder or a skin condition by topically applying the biophotonic compositions of the present disclosure. In certain such embodiments, a small quantity of the biophotonic composition, for example from 2-4 ml, may be applied to exposed areas of the skin, from a suitable container or applicator and, if necessary, it is then spread over the skin using the hand, fingers, or a suitable device. The amount of the biophotonic composition which is applied, the frequency of application and the period of use will vary widely depending upon the active levels of a given biophotonic composition and the level of regulation desired. The biophotonic composition is then illuminated by the user by putting on a device such as a home facial mask, thereafter activating the device's light source according to the manufacturer instructions.

Some skin disorders present various symptoms including redness, flushing, burning, scaling, pimples, papules, pustules, comedones, macules, nodules, vesicles, blisters, telangiectasia, spider veins, sores, surface irritations or pain, itching, inflammation, red, purple, or blue patches or discolorations, moles, and/or tumors.

Regulating skin condition may be practiced by applying a biophotonic composition of the present disclosure in the form of a skin lotion, cream, cosmetic, or the like which is intended to be left on the skin for an extended period for some aesthetic, prophylactic, therapeutic or other benefit (i.e., a “leave-on” composition). After applying the biophotonic composition to the skin, the leave-on biophotonic composition may be left on the skin for a period of at least about 10 minutes, or at least about 15 minutes, or at least about 30 minutes or greater than 30 minutes and not removed at all.

A pre-conditioning or post-conditioning composition may be applied to the target tissue, before, during or after the application of the presently disclosed composition. The conditioning composition may be a serum including for example an antioxidant such as Vitamin C, which can be reactive in the presence of lichen extracts (chromophores) and therefore advantageous to apply it to the target site separately.

Skin Aging and Rejuvenation

The dermis is the second layer of skin, containing the structural elements of the skin, the connective tissue. There are various types of connective tissue with different functions. Elastin fibers give the skin its elasticity, and collagen gives the skin its strength.

The junction between the dermis and the epidermis is an important structure. The dermal-epidermal junction interlocks forming finger-like epidermal ridges. The cells of the epidermis receive their nutrients from the blood vessels in the dermis. The epidermal ridges increase the surface area of the epidermis that is exposed to these blood vessels and the needed nutrients.

The aging of skin comes with significant physiological changes to the skin. The generation of new skin cells slows down, and the epidermal ridges of the dermal-epidermal junction flatten out. While the number of elastin fibers increases, their structure and coherence decrease. Also the amount of collagen and the thickness of the dermis decrease with the ageing of the skin.

Collagen is a major component of the skin's extracellular matrix, providing a structural framework. During the aging process, the decrease of collagen synthesis and insolubilization of collagen fibers contribute to a thinning of the dermis and loss of the skin's biomechanical properties.

The physiological changes to the skin result in noticeable aging symptoms often referred to as chronological-, intrinsic- and photo-ageing. The skin becomes drier, roughness and scaling increase, the appearance becomes duller, and most obviously fine lines and wrinkles appear. Other symptoms or signs of skin aging include, but are not limited to, thinning and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), inability to sweat sufficiently to cool the skin, unwanted facial hair, freckles, age spots, spider veins, rough and leathery skin, fine wrinkles that disappear when stretched, loose skin, a blotchy complexion.

The dermal-epidermal junction is a basement membrane that separates the keratinocytes in the epidermis from the extracellular matrix, which lies below in the dermis. This membrane consists of two layers: the basal lamina in contact with the keratinocytes, and the underlying reticular lamina in contact with the extracellular matrix. The basal lamina is rich in collagen type IV and laminin, molecules that play a role in providing a structural network and bioadhesive properties for cell attachment.

Laminin is a glycoprotein that only exists in basement membranes. It is composed of three polypeptide chains (alpha, beta and gamma) arranged in the shape of an asymmetric cross and held together by disulfide bonds. The three chains exist as different subtypes which result in twelve different isoforms for laminin, including Laminin-1 and Laminin-5.

The dermis is anchored to hemidesmosomes, specific junction points located on the keratinocytes, which consist of α-integrins and other proteins, at the basal membrane keratinocytes by type VII collagen fibrils. Laminins, and particularly Laminin-5, constitute the real anchor point between hemidesmosomal transmembrane proteins in basal keratinocytes and type VII collagen.

Laminin-5 synthesis and type VII collagen expression have been proven to decrease in aged skin. This causes a loss of contact between dermis and epidermis, and results in the skin losing elasticity and becoming saggy.

Recently another type of wrinkles, generally referred to as expression wrinkles, got general recognition. These wrinkles require loss of resilience, particularly in the dermis, because of which the skin is no longer able to resume its original state when facial muscles which produce facial expressions exert stress on the skin, resulting in expression wrinkles.

The present disclosure provides biophotonic compositions and methods for preventing, arresting, reversing, ameliorating, diminishing, reducing or improving a sign of aging, e.g., skin rejuvenation, in which a composition of the present disclosure is topically applied to skin in a cosmetically effective amount sufficient to prevent, arrest, reverse ameliorate, diminish, reduce or improve a sign of aging in skin. Exemplary signs of aging include, but are not limited to, facial lines, fine lines, wrinkles, crow's feet, dark eye circles, blemishes, age spots, stretch marks, or combinations thereof.

The present disclosure also provides biophotonic compositions and methods for improving the aesthetic appearance of skin, in which a composition of the present disclosure is topically applied to skin in a cosmetically effective amount sufficient to improve the aesthetic appearance of the skin. The improvements may relate to skin thickness, elasticity, resiliency, moisturization, tone, texture, radiance, luster, brightness, clarity, contour, firmness, tautness, suppleness, softness, sensitivity, pore size, or combinations thereof.

In certain embodiments, the biophotonic compositions and methods of the present disclosure promote collagen synthesis. In certain other embodiments, the biophotonic compositions and methods of the present disclosure may reduce, diminish, retard or even reverse one or more signs of skin aging including, but not limited to, appearance of fine lines or wrinkles, thin and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), inability to sweat sufficiently to cool the skin, unwanted facial hair, freckles, age spots, spider veins, rough and leathery skin, fine wrinkles that disappear when stretched, loose skin, or a blotchy complexion. In certain embodiments, the biophotonic compositions and methods of the present disclosure may induce a reduction in pore size, enhance sculpturing of skin subsections, and/or enhance skin translucence.

Acne and Acne Scar

The biophotonic compositions and methods of the present disclosure may be used to treat acne. As used herein, “acne” means a disorder of the skin caused by inflammation of skin glands or hair follicles. The compositions and methods of the disclosure can be used to treat acne at early pre-emergent stages or later stages where lesions from acne are visible. Mild to moderate acne can be treated with embodiments of the compositions and methods. Early pre-emergent stages of acne usually begin with an excessive secretion of sebum or dermal oil from the sebaceous glands located in the pilosebaceous apparatus. Sebum reaches the skin surface through the duct of the hair follicle. The presence of excessive amounts of sebum in the duct and on the skin tends to obstruct or stagnate the normal flow of sebum from the follicular duct, thus producing a thickening and solidification of the sebum to create a solid plug known as a comedone. In the normal sequence of developing acne, hyperkeratinazation of the follicular opening is stimulated, thus completing blocking of the duct. The usual results are papules, pustules, or cysts, often contaminated with bacteria, which cause secondary infections. Acne is characterized particularly by the presence of comedones, inflammatory papules, or cysts. The appearance of acne may range from slight skin irritation to pitting and even the development of disfiguring scars. Accordingly, the biophotonic biophotonic compositions and methods of the present disclosure can be used to treat one or more of skin irritation, pitting, development of scars, comedones, inflammatory papules, cysts, hyperkeratinazation, and thickening and hardening of sebum associated with acne, and also to impact upon the bacterial population of P. acnes resulting in the reduction of the bacterial population thereby reducing the manifestation of conditions relating to P. acnes, and hence having an antibacterial and/or bacterial reduction effect.

The biophotonic compositions and methods of the present disclosure may be used to treat various types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, and nodulocystic acne.

In some embodiments, the biophotonic compositions of the present disclosure are used in conjunction with systemic or topical antibiotic treatment. For example, antibiotics used to treat acne include tetracycline, erythromycin, minocycline, doxycycline, which may also be used with the compositions and methods of the present disclosure. The use of the can reduce the time needed for the antibiotic treatment or reduce the dosage.

Any one or more of the features of the previously described embodiments may be combined in any manner. Many variations of the subject matters will become apparent to those skilled in the art upon review of the specification. The following example is offered by way of illustration and not by way of limitation.

### EXAMPLES

The examples below are given so as to illustrate the practice of various embodiments of the present disclosure. They are not intended to limit or define the entire scope of this disclosure.

### Example 1: Preparation of the Biophotonic Compositions

For the preparation of the biophotonic compositions of the disclosure, the following methodology was utilized. The Rhizocarpon geographicum lichen extract was prepared by crushing the lichen into a semi-fine, homogeneous powder using a kitchen blender device. Thereafter, 1 gram of the particular lichen were then added to 5 mL of propylene glycol and the resulting solution was continuously stirred at low speed over the course of 5 days of continuous mixing. At the completion of the stirring period, the resulting product is left unfiltered with the particulate (solid) fraction remaining on the bottom of the mixing container while samples are withdrawn from the overlying liquid phase for analysis and inclusion into the biophotonic composition. The Xanthoparmelia scarbrosa lichen extract was prepared following the same protocol as described above. In certain situations, depending on the lichen species, different amount of propylene glycol to lichen powder ratio can be used. For example, extraction of lichen was carried out using the following ratios of lichen powdered to propylene glycol: (1) for Xanthoparmelia scarbrosa lichen, a 1:5 (wt:wt) ratio was used; (2) for Rhizocarpon geographicum lichen, a 1:5 (wt:wt) ratio was used; and (3) for Cetraria islandica lichen, a 1:3 (wt:wt) ratio was used. Furthermore, extraction of lichen may also be carried out in a 1:1 (wt:wt) ratio, or 1:2 (wt:wt) ratio, 1:4 (wt:wt), 1:5 (wt:wt), 1:6 (wt:wt), 1:7 (wt:wt), 1:8 (wt:wt), 1:9 (wt:wt), 1:10 (wt:wt) or 1:20 (wt:wt), 1:30 (wt:wt) ratio of the lichen powder to propylene glycol.

### Example 2: Exemplary Formulations of the Biophotonic Compositions

Composition a (without Lichen Extract)

| Ingredient | % w/w |
|---|---|
| SODIUM HYALURONATE | 0.22 |
| PURIFIED WATER | 98.61 |
| PHENOXYETHANOL | 0.30 |
| CARBOPOL 980 | 0.17 |
| SODIUM HYDROXIDE sol 32% w/w or HYDROCHLORIC | ~0.7 |
| ACID sol 15% w/w; qs pH 5.5 | |

Exemplary Formulation of a Biophotonic Composition comprising Composition A and lichen extract(s) (e.g., lichen extract(s) derived from Rhizocarpon geographicum, Xanthoparmelia scabrosa, Lecidella elaeochroma, Cetraria islandica, or Xanthoria parietina)

| Ingredient | % w/w | mg/g |
|---|---|---|
| SODIUM HYALURONATE | 0.22 | 2.2 |
| LICHEN EXTRACT IN PROPYLENE | 4.00 | 40 |
| GLYCOL | | |
| PURIFIED WATER | 95.31 | 953.1 |
| PHENOXYETHANOL | 0.30 | 3 |
| CARBOPOL 980 | 0.17 | 1.7 |
| SODIUM HYDROXIDE sol 32% w/w or | qs pH 5.5 | qs pH 5.5 |
| HYDROCHLORIC ACID sol 15% w/w | | |

### Example 3: Absorbance and Fluorescence of Lichen Extracts

This experiment was conducted to determine the absorbance and fluorescence profile of lichen extract A (derived from Rhizocarpon geographicum) and lichen extract B (derived from Lecidella elaeochroma or Xanthoria parietina). The fluorescence measurement was carried out in such a way that the intensity of the excitation light was similar to that of a home facial mask, emitting blue light.

In this experiment, eight samples were prepared and measured. These samples included: lichen extract A in propylene glycol; lichen extract B in propylene glycol (from three different batches B(1), B(2), and B(3)); lichen extract A in Composition A; lichen extracts B(1), B(2), and B(3) in composition A, respectively; and composition A. All lichen extracts in Composition A were 4% by volume of the composition. Samples were prepared in a cuvette and measured with a UV-Vis spectrometer to determine their optimal absorbance wavelengths. These samples were then measured in the same cuvette in a fluorimeter. The fluorimeter excitation slits were adjusted to 1.59 mm and 1.01 mm so that the power emitted from the laser at 440 nm was equal to 1.64 mW/cm², which is the same as the intensity of a home facial mask.

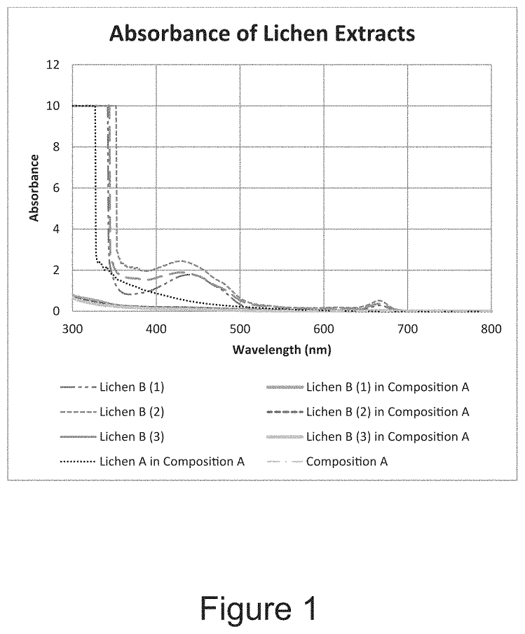

UV-vis absorbance of the lichen extracts showed some peaks of interest (FIG. 1). Each lichen B extract had the same absorbance with a small peak at 660 nm, a large peak at 440 nm, and a peak with maximum signal intensity at 300-350 nm. When these samples were placed in Composition A, the absorbance values decreased significantly, but no new peaks appeared. The sample of lichen A extract in Composition A had no other peaks except for a maximum signal intensity at 300 nm to 350 nm. The Composition A blank showed only a small peak from 300 nm to 350 nm.

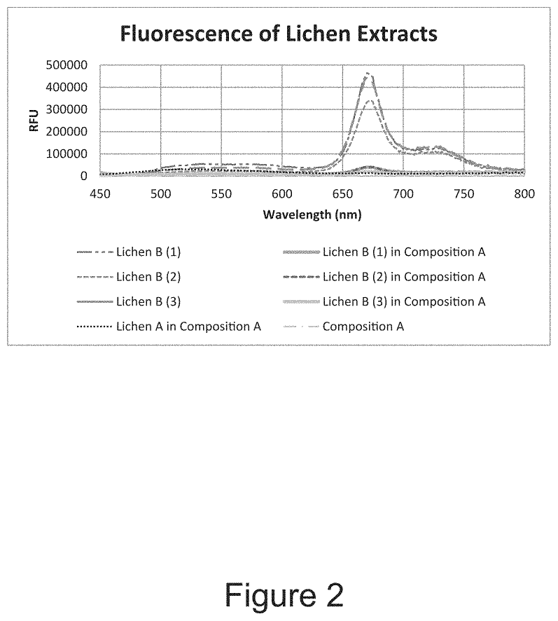

Fluorimeter measurement also resulted in some peaks of interest (FIG. 2). Each lichen extract B had the same emission profile with a long broad peak starting at 475 nm and ending around 625 nm, along with an intense peak at 675 nm, a smaller peak at 725 nm. When the lichen extracts were placed in Composition A, the fluorescence decreased significantly, though the profile itself remained the same. The sample of lichen A extract in Composition A had a small fluorescence at around 530 nm, which is very broad. The Composition A blank did not show any significant peaks.

In summary, the lichen extracts were measured for their absorbance and emission. The lichen B extracts showed a small absorbance at 660 nm and a large absorbance at 440 nm. The emission of lichen B extracts showed a broad peak from 475 nm to 625 nm, a very intense peak at 675 nm, and a smaller peak at 725 nm.

### Example 4: Method of Antibacterial Testing on 4% AXSE1 Lichen in Composition a with Low Intensity Light Source a and Low Intensity Light Source B Using Dilution-Neutralization Method

An assessment was performed to evaluate the anti-bacteria activities of the biophotonic composition comprising a lichen extract (AXSE1 lichen, extracted from Xanthoparmelia scabrosa) using different light sources. The light sources used for this experiment are: (1) low intensity light source A; and (2) low intensity light source B, intensity=5, blue LED).

P. acnes bacteria (ATCC 6919) were prepared from ATCC 2107 agar and incubated for 72 hours. Loop from ATCC 2107 agar was diluted into 5 ml of 0.9% saline. Serial dilutions up to 10⁻⁷ were performed and 10⁻⁵ to 10⁻⁷ was plated using 0.1 ml on ATCC 2107 agar in duplicate. These samples were to determine the initial concentration of bacteria used for the test.

0.2 ml of initial bacterial suspension (approximately 10⁷ to 10⁸) was added to 1.8 ml of treatment solution (placebo carrier gel or Composition A) and then well mixed together into 16×100 glass culture tubes (thereby the treatment solution was diluted by 1.11 fold). The glass culture tube was placed horizontally under the lamp.

Illuminating treatment of the P. acnes sample was carried out for 10 minutes with the low intensity light source A or and the low intensity light source B at approximately 2 cm to 3 cm distance from the bacteria sample. The chin part of the low intensity light source A was used, while the forehead part of the low intensity light source B was used during the illumination. The low intensity light source B was set at intensity 5 with blue LED light.

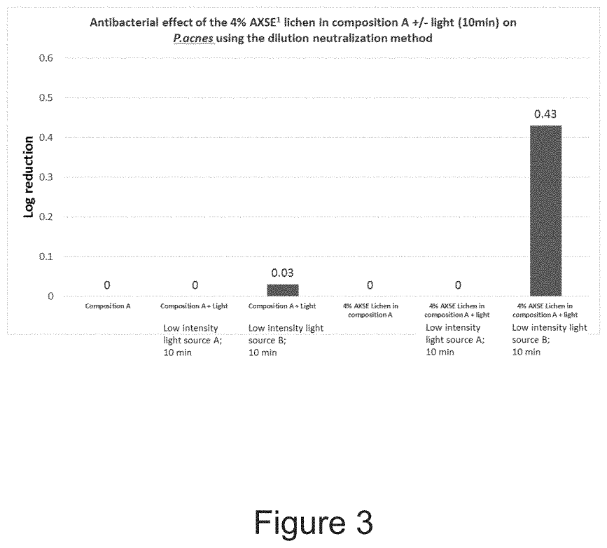

After the treatment, serials dilutions were performed in 0.9% saline. 0.100 ml of the dilutions was spread plated on ATCC 2107 agar in duplicate. All plates were incubated at 37° C., 72 hours under anaerobic conditions. Results were shown in FIG. 3. As shown in FIG. 3, a 0.43 log reduction of P. acnes was observed in the bacteria sample which was treated with 4% AXSE1 lichen in Composition A and illuminated for 10 minutes with low intensity light B. In comparison, when the bacteria sample was treated with just Composition A and illuminated under the same condition, only a 0.03 log reduction of P. acnes was observed.

### Example 5: Method for Determining Anti-Bacterial Activities of the BioPhotonic (BP) Solution with Low Intensity Light Source a Using Dilution-Neutralization Method

An assessment was performed to evaluate the anti-bacteria or bacteriostatic activities of the biophotonic composition comprising lichen extract. The BioPhotonic solution was prepared with the lichen extract (extracted from Rhizocarpon geographicum) and Composition A. P. acnes bacteria (ATCC 6919) were prepared from ATCC 2107 agar and incubated for 72 hours under anaerobic conditions. Loop from ATCC 2107 agar was diluted into 5 ml of 0.9% saline. Serial dilutions up to 10⁻⁷ were performed and 10⁻⁵ to 10⁻⁷ were plated using 0.1 ml on ATCC 2107 agar in duplicate. These results are to determine the initial concentration of bacteria used for the test.

0.2 ml of initial bacterial suspension (approximately 10⁷ to 10⁸), 0.2 ml of saline 0.9%, and 1.6 ml of treatment solution were mixed together in 16×100 glass culture tubes (thereby the treatment solution was diluted by 1.25 fold). The glass culture tube was placed horizontally under the lamp.

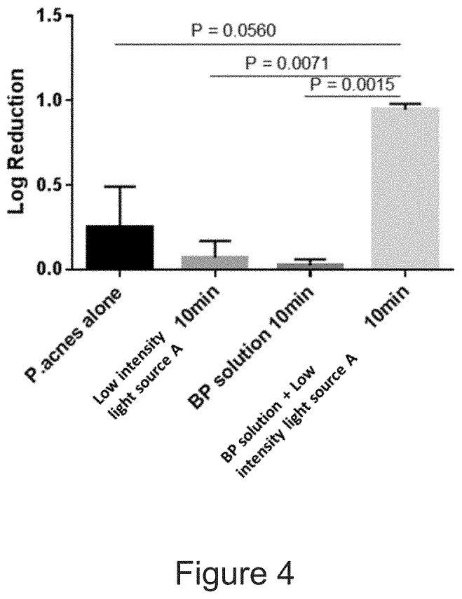

Illuminating treatment of the P. acnes sample was carried out for 10 minutes with the low intensity light source A at approximately 2 cm to 3 cm distance from the bacteria sample. The chin part of the low intensity light source A was used. After the treatment, 1 mL of the bacteria sample was added into 16×125 mm glass tube containing 6.95 ml of 0.9% saline and 0.05 ml of catalase. The catalase neutralizes hydrogen peroxide, H₂O₂. The neutralization step was carried out for 5 minutes (even if there was no H₂O₂ present, the neutralization step was performed in all samples). Serial dilutions were performed and 0.100 ml of the diluted solutions was spread on ATCC 2107 agar in duplicates. All plates were incubated at 37° C., minimum 72 hours and under anaerobic conditions. Results were shown in FIG. 4. As shown in FIG. 4, treatment of the bacteria sample with the BioPhotonic composition and illumination with a low intensity light yielded 1 (one) log reduction of P. acnes in the sample. In comparison, treatment of either the BioPhotonic or illumination with the low intensity light source A alone did not lead to significant reduction of P. acnes.

### Example 6: Clinical Treatment of Patients with Acne with BioPhotonic (BP) Lotion and Low Intensity Light Source A

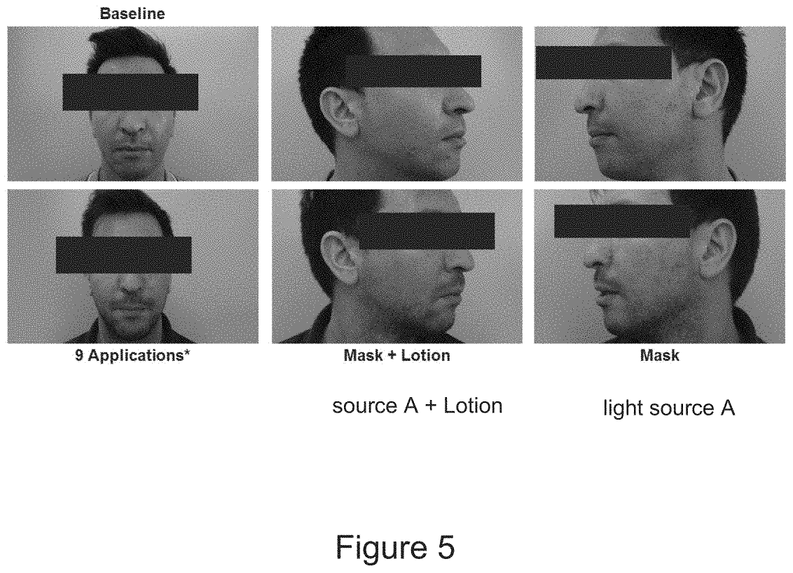

A clinical assessment was conducted to evaluate a use of the BioPhotonic composition of the present disclosure, when illuminated with a low intensity light source, to ameliorate an acne condition in a patient suffering from mild to moderate acne in comparison to use of the low intensity light source alone to ameliorate the patient's acne condition. BioPhotonic lotion was prepared with the lichen extract (extracted from Rhizocarpon geographicum) and Composition A. The BioPhotonic lotion of the assessment has the same composition as the BioPhotonic solution in Example 5. The assessment was carried out following a split face protocol with both a male and a female patient. The patient's face was cleaned before the treatment. The BioPhotonic lotion (about 1.5 mL) was applied on only one side (right side) of the patient's face and the other side (left side) was not applied with the BioPhotonic lotion for comparison. Both sides of the patients' face were concomitantly illuminated with the low intensity light source A once per day for 10 minutes each time (one application). The BioPhotonic lotion was left on the patient's face after the illumination was completed for each application. The male patient was treated for 9 applications (9 days, once per day for 10 minutes each time) and the female patient was treated for 7 applications (7 days, once per day for 10 minutes each time). Results of the assessment were shown in FIGS. 5A and 5B.

### Example 7: Method of Antibacterial Testing on Islandica bulgarie Lichen in Composition a with Low Intensity Light Source a Using Dilution-Neutralization Method

An assessment was performed to evaluate the anti-bacterial activity of the biophotonic composition comprising Islandica bulgarie lichen wherein the biophotonic composition is exposed to low intensity light source A (treated biophotonic composition) or not exposed to low intensity light source A (untreated biophotonic composition). The biophotonic composition (Composition A) comprising Islandica bulgarie lichen was prepared as set out in Example 1. The light sources used for this experiment was low intensity light source A. P. acnes bacteria (ATCC 6919) were prepared from ATCC 2107 agar and incubated for 72 hours under anaerobic conditions. Loop from ATCC 2107 agar was diluted into 5 ml of 0.9% saline. Serial dilutions up to 10⁻⁷ were performed and 10⁻⁵ to 10⁻⁷ were plated using 0.1 ml on ATCC 2107 agar in duplicate. These results are to determine the initial concentration of bacteria used for the test.

Light-Treated Biophotonic Composition:

A first 0.2 ml of initial bacterial suspension (approximately 10⁷ to 10⁸), 0.2 ml of saline 0.9%, and 1.6 ml of treatment solution (Composition A) were mixed together in 16×125 glass culture tubes. The glass culture tube was placed horizontally under the lamp. The bacterial suspension of the P. acnes sample was carried out for 10 minutes with the low intensity light source A at approximately 2 cm to 3 cm distance from the bacteria sample.

Untreated Biophotonic Composition:

A second 0.2 ml of initial bacterial suspension (approximately 10⁷ to 10⁸), 0.2 ml of saline 0.9%, and 1.6 ml of treatment solution (Composition A) were mixed together in 16×125 glass culture tubes. The glass culture tube was not placed under the lamp and was not illuminated with light source A.

1 mL of the light treated and untreated samples were added into 16×125 mm glass tube containing 7 ml of 0.9% saline. Serial dilutions were performed and 0.100 ml of the diluted solutions was spread on ATCC 2107 agar in duplicates. All plates were incubated at 37° C., minimum 72 hours and under anaerobic conditions.

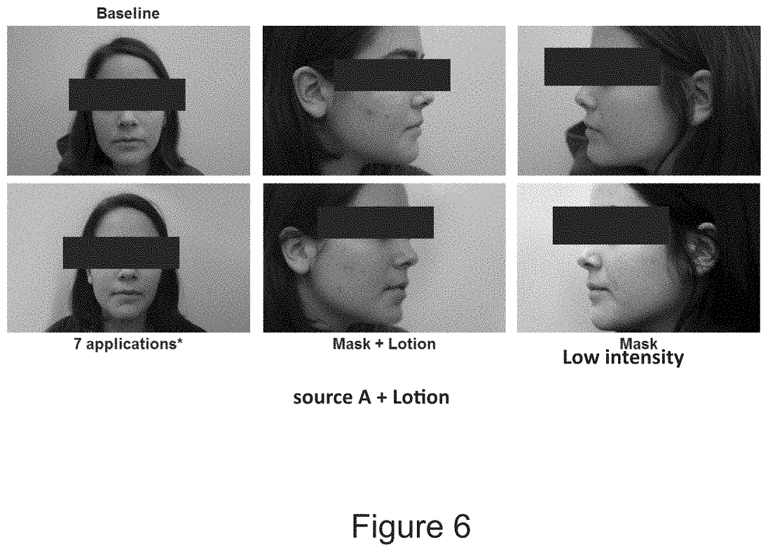

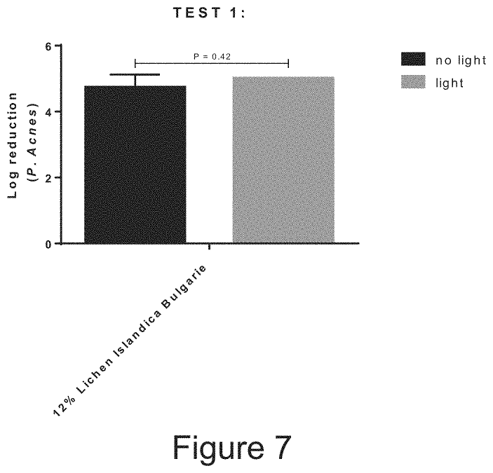

Results are shown in FIG. 6. The results indicate that both light treated Composition A and the untreated Composition A show similar antimicrobial activity against P. acnes. The results suggest that Islandica bulgarie lichen, by itself, controls bacterial activity (i.e., without light).

It should be appreciated that the disclosure is not limited to the particular embodiments described and illustrated herein but includes all modifications and variations falling within the scope of the subject matters as defined in the appended claims.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A biophotonic composition, comprising: a lichen extract and a carrier medium, wherein the lichen extract comprises at least one chromophore.

2. The biophotonic composition of claim 1, wherein the lichen extract is derived from a lichen selected from Rhizocarpon geographicum, Xanthoparmelia scabrosa, Lecidella elaeochroma, Cetraria islandica, Islandica bulgarie and Xanthoria parietina.

3. The biophotonic composition of claim 2, wherein the lichen extract is derived from Rhizocarpon geographicum.

4. The biophotonic composition of claim 2, wherein the lichen extract is derived from Xanthoparmelia scabrosa.

5. The biophotonic composition of claim 2, wherein the lichen extract is derived from Lecidella elaeochroma or Xanthoria parietina.

6. The biophotonic composition of claim 2, wherein the lichen extract is derived from Xanthoria parietina.

7. The biophotonic composition of claim 2, wherein the lichen extract is derived from Cetraria islandica.

8. The biophotonic composition of claim 1, wherein the carrier medium comprises one or more of a hydrophilic polymer, a hygroscopic polymer, or a hydrated polymer, or combinations thereof.

9. The biophotonic composition of claim 1, wherein the carrier medium comprises one or more of a synthetic polymer selected from a vinyl polymer, a polyoxyethylene-polyoxypropylene copolymer, poly(ethylene oxide), an acrylamide polymer, and derivatives or salts thereof.

10. The biophotonic composition of claim 1, wherein the carrier medium comprises carboxylic functional groups.

11. The biophotonic composition of claim 10, wherein the carrier medium comprises one or more of a vinyl polymer selected from polyacrylic acid, polymethacrylic acid, poly hydroxyethyl methacrylate, polyvinyl pyrrolidone, and polyvinyl alcohol.

12. (canceled)

13. The biophotonic composition of claim 1, wherein the carrier medium comprises one or more protein-based polymer.

14. The biophotonic composition of claim 13, wherein the protein-based polymer is selected from elastin, gelatin, and collagen.

15. The biophotonic composition of claim 1, wherein the carrier medium comprises one or more polysaccharide.

16. The biophotonic composition of claim 15, wherein the polysaccharide is selected from sodium hyaluronate, starch, chitosan, chitin, agar, an alginate, xanthan, carrageenan, guar gum, gellan gum, pectin, locust bean gum, hydroxypropyl cellulose, carboxymethyl cellulose, and combinations thereof.

17. The biophotonic composition of claim 1, wherein the carrier medium comprises at least one glycol.

18.-20. (canceled)

21. The biophotonic composition of claim 1, wherein the chromophore is a fluorescent chromophore.

22.-23. (canceled)

24. The biophotonic composition of claim 23, wherein the chromophore absorbs and/or emits light within the green, orange and yellow portions of the electromagnetic spectrum.

25.-26. (canceled)

27. The biophotonic composition of claim 1, wherein upon exposure to light, the composition emits at least 1.25×, 1.5×, 1.75× or 2× more red, yellow and/or orange light than a composition lacking the lichen extract.

28.-34. (canceled)

35. A method for biophotonic treatment of a skin disorder, comprising:
applying the biophotonic composition according to claim 1 to a target skin tissue, and
illuminating said biophotonic composition with low intensity light.

36.-80. (canceled)
